(19) United States
(12) Patent Application Publication
(10) Pub. No.: US 2008/0139493 A1
(43) Pub. Date: Jun. 12, 2008

(54) Bi- or Tetra-Guanidino-Biphenyl Compounds as Small Molecule Carriers

(76) Inventors: David Selwood, London (GB); Cristina Visintin, London (GB); Heike Laman, Cambridge (GB); Masahiro Okuyama, Osaka (JP)

Correspondence Address: NIXON & VANDERHYE, PC, 901 NORTH GLEBE ROAD, 11TH FLOOR, ARLINGTON, VA, 22203, US

(21) Appl. No.: 11/629,400

(22) PCT Filed: Jun. 17, 2005
(86) PCT No.: PCT/GB05/02399

§ 371 Date: Nov. 9, 2007

(30) Foreign Application Priority Data
Jun. 17, 2004 (GB) 0413613.1

(51) Int. Cl. A61K 31/4402; C07D 311/82; C07D 213/71; A61K 47/22; A61P 43/00; A61K 31/7088; A61K 31/352

(52) U.S. Cl. 514/44; 549/388; 546/294; 514/788; 514/454; 514/347

(57) ABSTRACT

The invention relates to compounds of formula I, or pharmaceutically acceptable salts thereof,

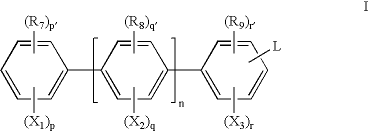

wherein X₁, X₂ and X₃ are each independently

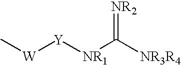

where Y is an alkylene, alkenylene or alkynylene group, each of which may be optionally substituted with one or more substituents selected from alkyl, halo, CF₃, OH, alkoxy, NH₂, CN, NO₂ and COOH; W is absent or is O, S or NH; R₁, R₂, R₃ and R₄ are each independently selected from H, alkyl, aryl and a protecting group P₁;
R₇, R₈ and R₉ are each independently selected from H, alkyl, halo, CF₃, OH, alkoxy, NH₂, CN, NO₂ and COOH; q and r are each independently 1, 2, 3 or 4; q′ and r′ are each independently 0, 1, 2 or 3, where q+q′ and r+r′ each equal 4; p is 1, 2, 3, 4 or 5, and p′ is 0, 1, 2, 3 or 4, where p+p′ is 5; n is 0, 1, 2, 3 . . . 6;
L is (Z)ₘNR₅R₆ where Z is a hydrocarbyl group and m is 0 or 1;
   where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore

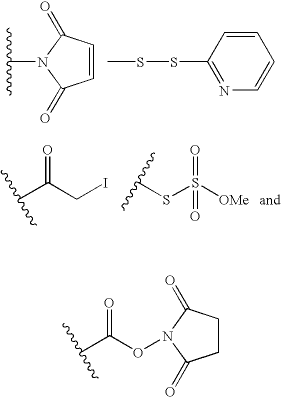

or R₅, R₆ and the nitrogen to which they are attached together form

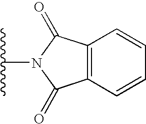

The present invention relates to small molecule carriers (SMCs). More specifically, the invention relates to SMCs that are useful for the in vitro and in vivo delivery of various cargo moieties into cells.

### BACKGROUND TO THE INVENTION

Over recent years, studies have shown that a variety of peptides, many of which are present in viral proteins, have the ability to cross biological membranes in various different cell types. These peptides, known as “protein transduction domains” (PTDs), can be linked to a wide variety of molecules with limited ability to cross membranes, (e.g., peptides, proteins, DNA), thereby enabling them to traverse biological membranes. Studies have shown that PTD fusion molecules introduced into mice exhibit delivery to all tissues, including the traversal of the blood-brain barrier [Schwarze, S R., Dowdy, S F., Trends Pharmacol. Sci, 2000, 21, 45]. Similar basic peptides are known to have anti-bacterial activity against MDR forms.

Most therapeutic drugs are limited to a relatively narrow range of physical properties. By way of example, they must be sufficiently polar for administration and distribution, but sufficiently non-polar so as to allow passive diffusion through the relatively non-polar bilayer of the cell. As a consequence, many promising drug candidates (including many peptide drugs) fail to advance clinically because they fall outside of this range, proving to be either too non-polar for administration and distribution, or too polar for passive cellular entry. A novel approach to circumvent this problem is to covalently tether these potential drugs to PTDs. However, it is very costly and time consuming to prepare such peptide-PTDs and their peptide structure often renders them susceptible to rapid degradation by cellular enzymes.

The present invention seeks to provide small molecule carriers (SMCs or “molecular tugs”) that are more amenable than peptide-PTDs due to their ease of preparation and their in vivo stability by virtue of their resistance to cellular enzymes that degrade peptides.

### STATEMENT OF INVENTION

A first aspect of the invention relates to a compound of formula I, or a pharmaceutically acceptable salt thereof,

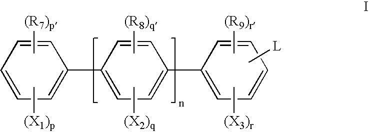

wherein
X₁, X₂ and X₃ are each independently

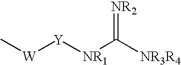

where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
   R₇, R₈ and R₉ are each independently selected from H, alkyl, halo, CF₃, OH, alkoxy, NH₂, CN, NO₂ and COOH;
   q and r are each independently 1, 2, 3 or 4;
   q′ and r′ are each independently 0, 1, 2 or 3, where q+q′ and r+r′ each equal 4;
   p is 1, 2, 3, 4 or 5, and p′ is 0, 1, 2, 3 or 4, where p+p′ is 5;
   n is 0 or 1, 2, 3 . . . 6;
   L is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore,

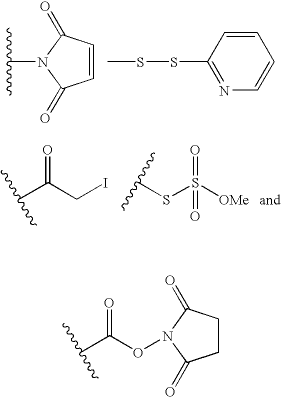

or R₅, R₆ and the nitrogen to which they are attached together form

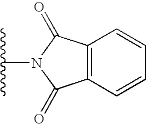

A second aspect of the invention relates to a conjugate comprising a compound of formula I as defined above linked to a cargo moiety.

A third aspect of the invention relates to a pharmaceutical composition comprising a compound of formula I as defined above, or a conjugate as defined above, and a pharmaceutically acceptable excipient, diluent or carrier

A fourth aspect of the invention relates to a compound of formula I as defined above, or a conjugate as defined above, for use in medicine.

A fifth aspect of the invention relates to a delivery system comprising a drug moiety linked to a carrier moiety, wherein the carrier moiety is a compound of formula I as defined above.

A sixth aspect of the invention relates to a conjugate comprising the reaction product of:

(i) a compound of formula Ic, or a pharmaceutically acceptable salt thereof,

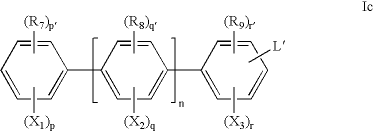

wherein
X₁, X₂ and X₃ are each independently

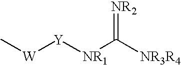

where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
   R₇, R₈ and R₉ are each independently selected from H, alkyl, halo, CF₃, OH, alkoxy, NH₂, CN, NO₂ and COOH;
   q and r are each independently 1, 2, 3 or 4;
   q′ and r′ are each independently 0, 1, 2 or 3, where q+q′ and r+r′ each equal 4;
   p is 1, 2, 3, 4 or 5, and p′ is 0, 1, 2, 3 or 4, where p+p′ is 5;
   n is 0 or 1, 2, 3 . . . 6;
   L′ is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore

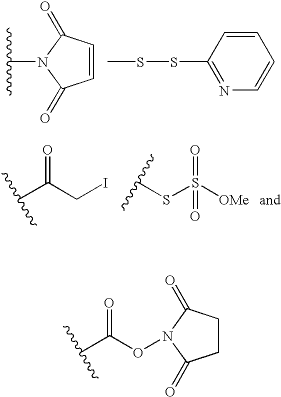

and
(ii) a cargo moiety selected from a protein, a peptide, an antibody or a drug.

A seventh aspect of the invention relates to a process for preparing a conjugate as defined above.

An eighth aspect of the invention relates to a method for introducing a cargo moiety into a cell, said method comprising contacting said cell with a conjugate as defined above.

A ninth aspect of the invention relates to a process for preparing a compound of formula I as defined above.

A tenth aspect of the invention relates to a compound of formula Id, or a pharmaceutically acceptable salt thereof,

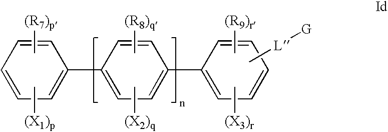

wherein
X₁, X₂ and X₃ are each independently

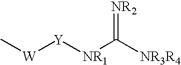

where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
   R₇, R₈ and R₉ are each independently selected from H, alkyl, halo, CF₃, OH, alkoxy, NH₂, CN, NO₂ and COOH;
   q and r are each independently 1, 2, 3 or 4;
   q′ and r′ are each independently 0, 1, 2 or 3, where q+q′ and r+r′ each equal 4;
   p is 1, 2, 3, 4 or 5, and p′ is 0, 1, 2, 3 or 4, where p+p′ is 5;
   n is 0 or 1, 2, 3 . . . 6;
   L″ is -(Z)ₘNR₅— where Z is a hydrocarbyl group and m is 0 or 1;
where R₅ is H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore,

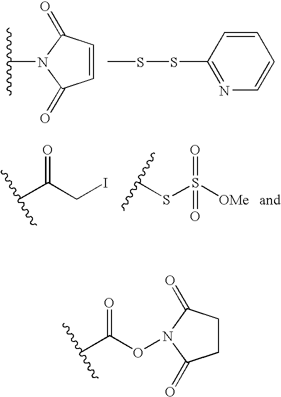

G is a cargo moiety.

### DETAILED DESCRIPTION

As used herein, the term “hydrocarbyl” refers to a saturated or unsaturated, straight-chain, branched, or cyclic group comprising at least C and H that may optionally comprise one or more other suitable substituents. Examples of such substituents may include halo, alkoxy, hydroxy, CF₃, CN, amino, COOH, nitro or a cyclic group. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon. Preferably, the hydrocarbyl group is an aryl or alkyl group.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl. Suitable substituents include halo, CF₃, OH, alkoxy, NH₂, CN, NO₂ and COOH. The term “alkylene” should be construed accordingly.

As used herein, the term “aryl” refers to a substituted (mono- or poly-) or unsubstituted monoaromatic or polyaromatic system, wherein said polyaromatic system may be fused or unfused. Preferably, the term “aryl” is includes groups having from 6 to 10 carbon atoms, e.g. phenyl, naphthyl etc. The term “aryl” is synonymous with the term “aromatic”. Suitable substituents include alkyl, halo, CF₃, OH, alkoxy, NH₂, CN, NO₂ and COOH. Preferably, the aryl group is an optionally substituted phenyl group.

As used herein, the term “alkenyl” refers to a group containing one or more carbon-carbon double bonds, which may be branched or unbranched, substituted (mono- or poly-) or unsubstituted. Preferably the alkenyl group is a C₂₋₂₀ alkenyl group, more preferably a C₂₋₁₅ alkenyl group, more preferably still a C₂₋₁₂ alkenyl group, or preferably a C₂₋₆ alkenyl group, more preferably a C₂₋₃ alkenyl group. Suitable substituents include alkyl, halo, CF₃, OH, alkoxy, NH₂, CN, NO₂ and COOH. The term “alkenylene” should be construed accordingly.

As used herein, the term “alkynyl” refers to a carbon chain containing one or more triple bonds, which may be branched or unbranched, and substituted (mono- or poly-) or unsubstituted. Preferably the alkynyl group is a C₂₋₂₀ alkynyl group, more preferably a C₂₋₁₅ alkynyl group, more preferably still a C₂₋₁₂ alkynyl group, or preferably a C₂₋₆ alkynyl group or a C₂₋₃ alkynyl group. Suitable substituents include alkyl, halo, CF₃, OH, alkoxy, NH₂, CN, NO₂ and COOH. The term “alkynylene” should be construed accordingly.

As used herein, the term “chromophore” refers to any functional group that absorbs light, giving rise to colour. Typically, the term refers to a group of associated atoms which can exist in at least two states of energy, a ground state of relatively low energy and an excited state to which it may be raised by the absorption of light energy from a specified region of the radiation spectrum. Often, the group of associated atoms contains delocalised electrons.

For compounds of formula I, p, q and r are each independently 1, 2, 3 or 4.

In a preferred embodiment of the invention, Y is a C₁₋₁₀ alkylene group, a C₂₋₁₀ alkenylene group or a C₂₋₁₀ alkynylene group.

In a preferred embodiment, W is O.

More preferably, Y is a C₁₋₁₂ alkylene group, more preferably a C₁₋₁₀ alkylene group, even more preferably a C₁₋₆ alkylene group, and more preferably still, CH₂CH₂.

Preferably, m is 1 and Z is an alkylene group, more preferably, a C₁₋₁₂ alkylene group, more preferably still a C₁₋₁₀ alkylene group, even more preferably a C₁₋₆ alkylene group. More preferably, Z is a CH₂ group.

Preferably, one of R₅ and R₆ is H and the other is selected from H, CO(CH₂)ⱼQ₁ or C═S(NH)(CH₂)ₖQ₂, or R₅, R₆ and the nitrogen to which they are attached together form

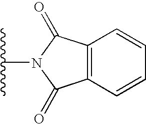

In one preferred embodiment, L is selected from the following: CH₂NH₂, CH₂NHCOCH₂CH₂COOH

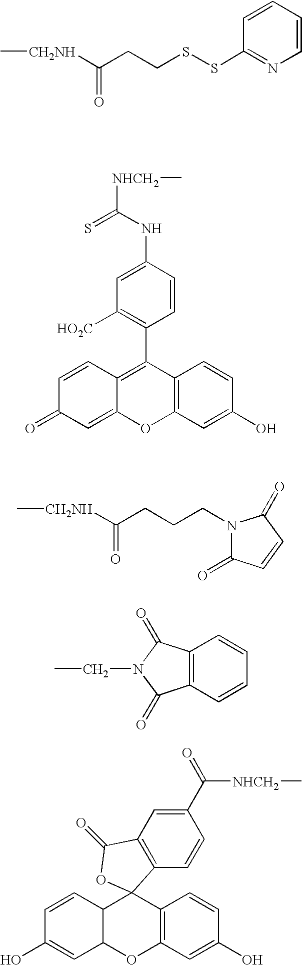

Preferably, R₁, R₂, R₃ and R₄ are each independently selected from H, or a protecting group P₁.

More preferably, R₁, R₂, R₃ and R₄ are each independently selected from H, or a butyloxycarbonyl (Boc) protecting group.

Preferably, p, q and r are each independently 1 or 2.

In one preferred embodiment, p, q and r are all equal to 1.

In another preferred embodiment, p, q and r are all equal to 2.

Preferably, R₇, R₈ and R₉ are all H.

In one particularly preferred embodiment, X₁, X₂ and X₃ are the same and are all

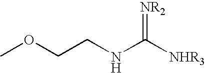

where R₂ and R₃ are each independently H or a Boc protecting group.

In one preferred embodiment, n is 0 or 1.

In a more preferred embodiment, n is 0.

In a more preferred embodiment, the compound of the invention is of formula Ia or Ib

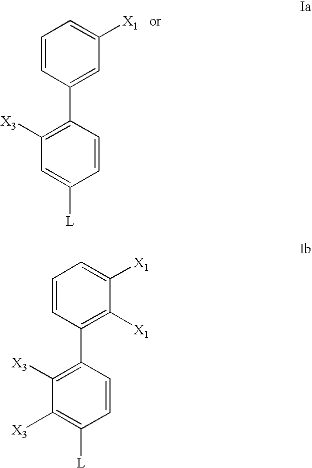

More preferably, X₁ and X₃ are the same and are both

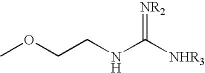

where R₂ and R₃ are each independently H or a Boc protecting group.

In one especially preferred embodiment, the compound of the invention is selected from the following:

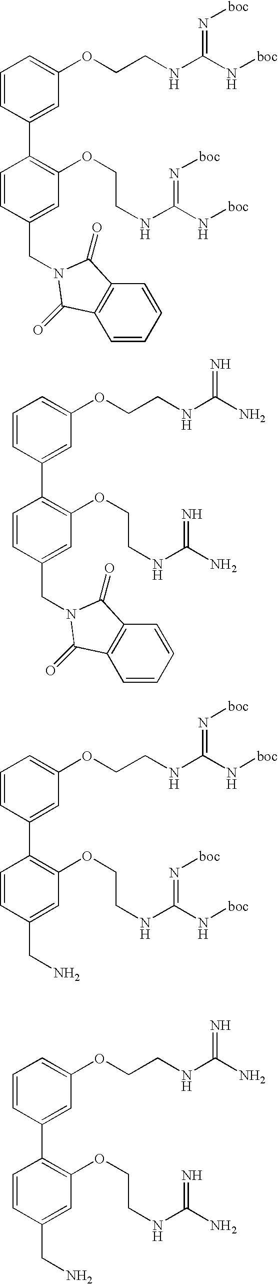

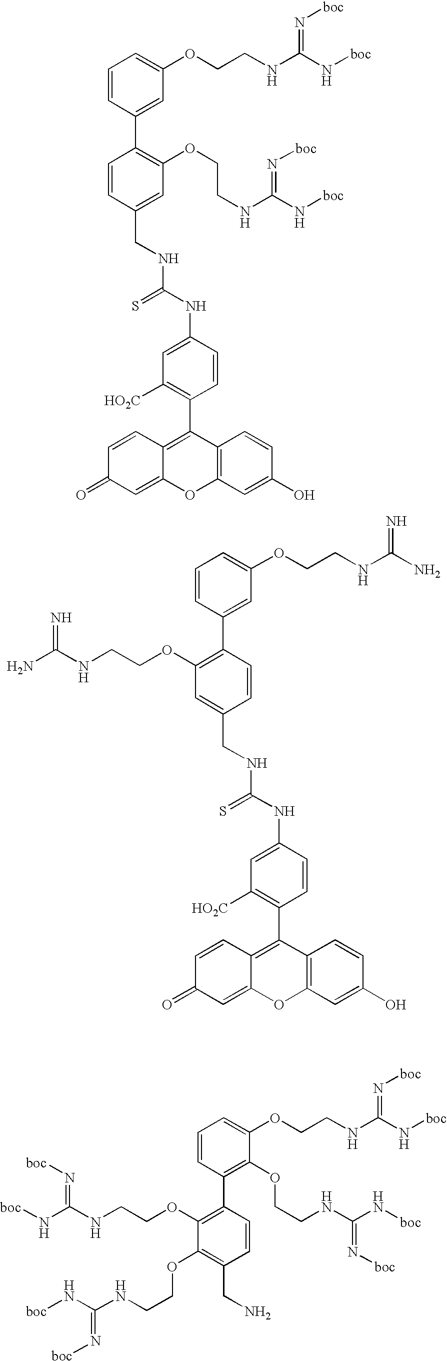

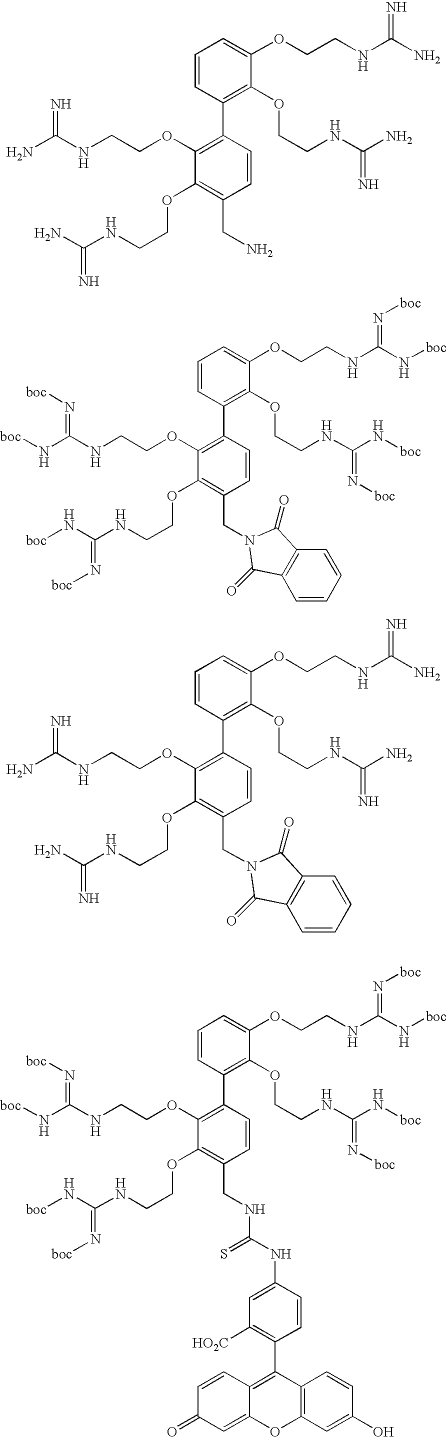

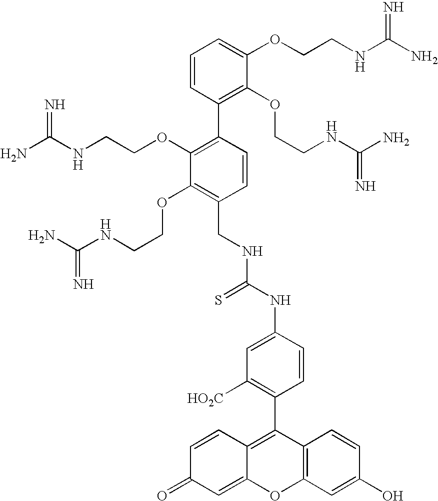

One preferred embodiment of the invention relates to a compound of formula Ie, or a pharmaceutically acceptable salt thereof,

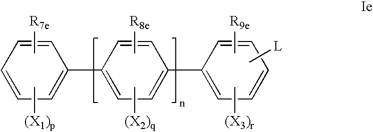

wherein
X₁, X₂ and X₃ are each independently

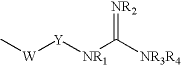

where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
   R₇ₑ, R₈ₑ and R₉ₑ are each independently absent or selected from alkyl, halo, CF₃, OH, alkoxy, NH₂, CN, NO₂ and COOH;
   p, q and r are each independently 1, 2, 3 or 4;
   n is 0 or 1, 2, 3 . . . 6;
   L is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore,

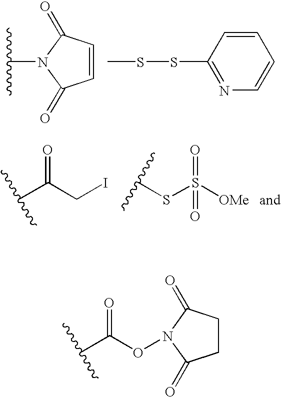

or R₅, R₆ and the nitrogen to which they are attached together form

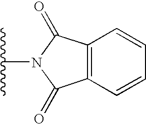

The skilled person will appreciate that when R₈ₑ and R₉ₑ are present, q and r may each independently be 1, 2 or 3, whereas when R₇ₑ is present, p may be 1, 2, 3 or 4.

Preferably, for compounds of formula Ie, R₇ₑ, R₈ₑ and R₉ₑ are absent.

Another aspect of the invention relates to a compound of formula If, or a pharmaceutically acceptable salt thereof,

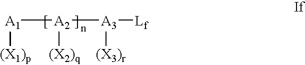

wherein X₁, X₂, X₃, p, q, r and n are as defined hereinabove for compounds of formula I;
each of A₁, A₂ and A₃ is independently a phenyl group optionally substituted by one or more additional substituents selected from alkyl, halo, CF₃, OH, alkoxy, NH₂, CN, NO₂ and COOH;
L_{f} is a linker group, preferably as defined for L above.

Preferred definitions of X₁, X₂, X₃, p, q, r and n are as defined hereinabove for compounds of formula I.

Preferably, A₁, A₂ and A₃ are the same.

### Conjugate

A second aspect of the invention relates to a conjugate comprising a compound of formula I, Ie or If as defined above linked to a cargo moiety.

Preferred X₁₋₃, Y, Z, R₁₋₉, N, j, k, l, p, q, r, n groups are as defined above for said first aspect.

In one embodiment, the conjugate comprises the reaction product of a compound of formula Ic or If as defined above and a cargo moiety.

The cargo moiety may comprise oligonucleotides, nucleotides, proteins, peptides, biologically active compounds, diagnostic agents, or combinations thereof.

The cargo moiety may be directly or indirectly linked to the carrier moiety. In the embodiment wherein the cargo moiety is indirectly linked to the carrier, the linkage may be by an intermediary bonding group such as a sulphydryl or carboxyl group or any larger group, all such linking groups are herein referred to as linker moieties as discussed below. Preferably, the carrier and cargo moieties are linked directly.

Examples of suitable oligonucleotide cargo moieties include genes, gene fragments, sequences of DNA, cDNA, RNA, nucleotides, nucleosides, heterocyclic bases, synthetic and non-synthetic, sense or anti-sense oligonucleotides including those with nuclease resistant backbones etc. or any of the above incorporating a radioactive label, that are desired to be delivered into a cell or alternatively to be delivered from a cell to its exterior. Preferably, the oligonucleotide cargo moiety is a gene or gene fragment.

Examples of suitable protein or peptide cargo moieties include; proteins, peptides, and their derivatives such as: antibodies and fragments thereof; cytokines and derivatives or fragments thereof, for example, the interleukins (IL) and especially the IL-1, IL-2, IL-3, IL-4, IL-5, IL-6, IL-7, IL-8, IL-9, IL-10, IL-11 and IL-12 subtypes thereof; colony stimulating factors, for example granulocyte-macrophage colony stimulating factor, granulocyte-colony stimulating factor (alpha and beta forms), macrophage colony stimulating factor (also known as CSF-1); haemopoietins, for example erythropoietin, haemopoietin-alpha and kit-ligand (also known as stem cell factor or Steel factor); interferons (IFNS), for example IFN-α, IFN-β and IFN-γ; growth factors and bifunctional growth modulators, for example epidermal growth factor, platelet derived growth factor, transforming growth factor (alpha and beta forms), amphiregulin, somatomedin-C, bone growth factor, fibroblast growth factors, insulin-like growth factors, heparin binding growth factors and tumour growth factors; differentiation factors and the like, for example macrophage differentiating factor, differentiation inducing factor (DIF) and leukaemia inhibitory factor; activating factors, for example platelet activating factor and macrophage activation factor; coagulation factors such as fibrinolytic/anticoagulant agents including heparin and proteases and their pro-factors, for example clotting factors VII, VIII, IX, X, XI and XII, antithrombin III, protein C, protein S, streptokinase, urokinase, prourokinase, tissue plasminogen activator, fibrinogen and hirudin; peptide hormones, for example insulin, growth hormone, gonadotrophins, follicle stimulating hormone, leutenising hormone, growth hormone releasing hormone and calcitonin; enzymes such as superoxide dismutase, glucocerebrosidase, asparaginase and adenosine deaminase; vaccines or vaccine antigens such as, for example hepatitis-B vaccine, malaria vaccine, melanoma vaccine and HIV-1 vaccine; transcription factors and transcriptional modulators.

Examples of a suitable non-nucleotide/proteinaceous biologically active cargo moieties are drug moieties selected from cytotoxic agents, anti-neoplastic agents, anti-hypertensives, cardioprotective agents, anti-arrhythmics, ACE inhibitors, anti-inflammatory's, diuretics, muscle relaxants, local anaesthetics, hormones, cholesterol lowering drugs, anti-coagulants, anti-depressants, tranquilizers, neuroleptics, analgesics such as a narcotic or anti-pyretic analgesics, anti-virals, anti-bacterials, anti-fungals, bacteriostats, CNS active agents, anti-convulsants, anxiolytics, antacids, narcotics, antibiotics, respiratory agents, anti-histamines, immunosuppressants, immunoactivating agents, nutritional additives, anti-tussives, diagnostic agents, emetics and anti-emetics, carbohydrates, glycosoaminoglycans, glycoproteins and polysaccharides; lipids, for example phosphatidyl-ethanolamine, phosphtidylserine and derivatives thereof; sphingosine; steroids; vitamins; antibiotics including lantibiotics; bacteristatic and bactericidal agents; antifungal, anthelminthic and other agents effective against infective agents including unicellular pathogens; small effector molecules such as noradrenalin, alpha adrenergic receptor ligands, dopamine receptor ligands, histamine receptor ligands, GABA/benzodiazepine receptor ligands, serotonin receptor ligands, leukotrienes and triodothyronine; cytotoxic agents such as doxorubicin, methotrexate and derivatives thereof.

In one preferred embodiment, the cargo moiety is selected from a protein, a peptide, an antibody and a drug.

In another preferred embodiment the cargo moiety is protein A, a bacterially derived protein that binds strongly to conventional antibodies.

Previous studies have demonstrated that a fusion protein containing the protein transduction domain of HIV-1 TAT and the B domain of staphylococcal protein A can be used to internalise antibodies into mammalian cells [Mie et al, Biochemical and Biophysical Research Communications 310 (2003); 730-734].

Preferably, the compound of formula I is linked to commercially available (natural) protein A via a lysine NH₂ group of protein A.

In one preferred embodiment, the conjugate of the invention is the reaction product of a protein with a compound of formula Ic as shown above wherein L′ is (Z)ₘNR₅R₆ where Z is a hydrocarbyl group and m is 0 or 1; where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore

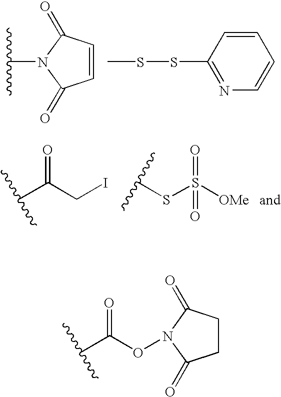

In one particularly preferred embodiment of the invention, the conjugate is the reaction product of a protein (such as for example, protein A) and a compound of formula Ic as shown above wherein L′ is (Z)ₘNR₅R₆ where Z is a hydrocarbyl group and m is 0 or 1; where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently

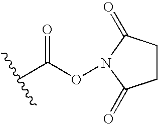

In an alternative preferred embodiment, a cysteine residue may be engineered into the protein to allow conjugation to said compound of formula Ic. Further details on the preparation of cysteine modified proteins may be found in Neisler et al [Bioconjugate Chem. 2002, 13, 729-736].

Preferably, the cargo moiety is covalently attached to the L group of said compound of formula I, Ie or If.

In one preferred embodiment, the cargo moiety is directly linked to the carrier moiety.

In another preferred embodiment, the cargo moiety is indirectly linked to the carrier moiety by means of a linker moiety.

Direct linkage may occur through any convenient functional group on the cargo moiety, such as a hydroxy, carboxy or amino group. Indirect linkage will occur through a linking moiety. Suitable linking moieties include bi- and multi-functional alkyl, aryl, aralkyl or peptidic moieties, alkyl, aryl or aralkyl aldehydes acids esters and anhydrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides and the like. The functional group on the linker moiety used to form covalent bonds between the compound of formula I and the cargo moiety may be, for example, amino, hydrazino, hydroxyl, thiol, maleimido, carbonyl, and carboxyl groups, etc. The linker moiety may include a short sequence of from 1 to 4 amino acid residues that optionally includes a cysteine residue through which the linker moiety bonds to the compound of formula I. Alternatively, the compound of formula I and the cargo moiety may be linked by leucine zippers, dimerisation domains, or an avidin/biotin linker.

In one preferred embodiment, the cargo moiety is selected from a recombinant antibody, a Fab fragment, a F(ab′)₂ fragment, a single chain Fv, a diabody, a disulfide linked Fv, a single antibody domain and a CDR.

As used herein, the term “CDR” or “complementary determining region” refers to the hypervariable regions of an antibody molecule, consisting of three loops from the heavy chain and three from the light chain, that together form the antigen-binding site. By way of example, the antibody may be selected from Herceptin, Rituxan, Theragyn (Pemtumomab), Infliximab, Zenapex, Panorex, Vitaxin, Protovir, EGFR1 or MFE-23. In one preferred embodiment, the cargo moiety is a genetically engineered fragment selected from a Fab fragment, a F(ab′)₂ fragment, a single chain Fv, or any other antibody-derived format.

Conventionally, the term “Fab fragment” refers to a protein fragment obtained (together with Fc and Fc′ fragments) by papain hydrolysis of an immunoglobulin molecule. It consists of one intact light chain linked by a disulfide bond to the N-terminal part of the contiguous heavy chain (the Fd fragment). Two Fab fragments are obtained from each immunoglobulin molecule, each fragment containing one binding site. In the context of the present invention, the Fab fragment may be prepared by gene expression of the relevant DNA sequences.

Conventionally, the term “F(ab′)₂” fragment refers to a protein fragment obtained (together with the pFc′ fragment) by pepsin hydrolysis of an immunoglobulin molecule. It consists of that part of the immunoglobulin molecule N-terminal to the site of pepsin attack and contains both Fab fragments held together by disulfide bonds in a short section of the Fc fragment (the hinge region). One F(ab′)₂ fragment is obtained from each immunoglobulin molecule; it contains two antigen binding sites, but not the site for complement fixation. In the context of the present invention, the F(ab′)₂ fragment may be prepared by gene expression of the relevant DNA sequences.

As used herein, the term “Fv fragment” refers to the N-terminal part of the Fab fragment of an immunoglobulin molecule, consisting of the variable portions of one light chain and one heavy chain. Single-chain Fvs (about 30 KDa) are artificial binding molecules derived from whole antibodies, but which contain the minimal part required to recognise antigen.

In another preferred embodiment, the cargo moiety is a synthetic or natural peptide, a growth factor, a hormone, a peptide ligand, a carbohydrate or a lipid.

The cargo moiety can be designed or selected from a combinatorial library to bind with high affinity and specificity to a target antigen. Typical affinities are in the 10⁻⁶ to 10⁻¹⁵ M K_{d} range. Functional amino acid residues present in the cargo moiety may be altered by site-directed mutagenesis where possible, without altering the properties of the cargo moiety. Examples of such changes include mutating any free surface thiol-containing residues (cysteine) to serines or alanines, altering lysines and arginines to asparagines and histidines, and altering serines to alanines.

Another embodiment of the invention provides a conjugate comprising the reaction product of:

(i) a compound of formula Ic, or a pharmaceutically acceptable salt thereof,

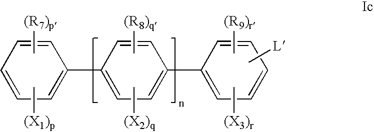

wherein
X₁, X₂ and X₃ are each independently

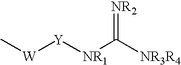

where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
   R₇, R₈ and R₉ are each independently selected from H, alkyl, halo, CF₃, OH, alkoxy, NH₂, CN, NO₂ and COOH;
   q and r are each independently 1, 2, 3 or 4;
   q′ and r′ are each independently 0, 1, 2 or 3, where q+q′ and r+r′ each equal 4;
   p is 1, 2, 3, 4 or 5, and p′ is 0, 1, 2, 3 or 4, where p+p′ is 5;
   n is 0, 1, 2, 3 . . . 6;
   L′ is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore

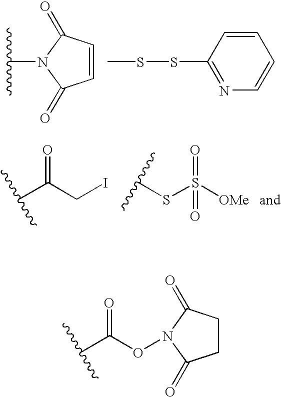

and
(ii) a cargo moiety selected from a protein, a peptide, an antibody or a drug.

Preferred X₁₋₃, Y, Z, R₁₋₉, N, j, k, l, p, q, r, n groups are as defined above for said first aspect.

Another aspect of the invention relates to a compound of formula Id, or a pharmaceutically acceptable salt thereof,

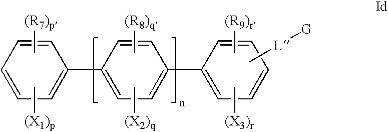

wherein
X₁, X₂ and X₃ are each independently

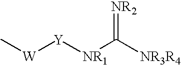

where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
   R₇, R₈ and R₉ are each independently selected from H, alkyl, halo, CF₃, OH, alkoxy, NH₂, CN, NO₂ and COOH;
   q and r are each independently 1, 2, 3 or 4;
   q′ and r′ are each independently 0, 1, 2 or 3, where q+q′ and r+r′ each equal 4;
   p is 1, 2, 3, 4 or 5, and p′ is 0, 1, 2, 3 or 4, where p+p′ is 5;
   n is 0, 1, 2, 3 . . . 6;
   L″ is -(Z)ₘNR₅— where Z is a hydrocarbyl group and m is 0 or 1;
where R₅ is H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore

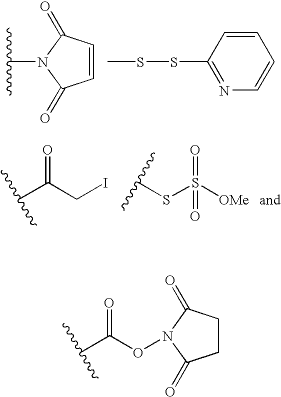

G is a cargo moiety. Preferably, the cargo moiety is as defined hereinabove.

Another aspect of the invention relates to a compound of formula Ig,

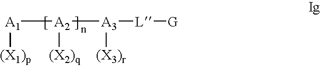

wherein A₁, A₂ and A₃, X₁, X₂, X₃, L″, G, p, q, r and n are as defined hereinabove.

Preferred X₁₋₃, Y, Z, R₁₋₉, N, j, k, l, p, q, r, n groups are as defined above for said first aspect.

Preferably, for this embodiment of the invention, L″ is -(Z)ₘNH.

### Delivery System

Another aspect of the invention relates to a delivery system comprising a drug moiety linked to a carrier moiety, wherein the carrier moiety is a compound of formula I, Ie or If as defined above.

In one embodiment, the delivery system comprises the reaction product of a compound of formula I, Ie or If as defined above and a drug moiety.

Preferably, the delivery system is therapeutically active in its intact state.

Preferably, the drug moiety is selected from those listed hereinbefore as suitable cargo moieties.

More preferably, the drug moiety is derived from a cytotoxic drug.

More preferably, the drug moiety is selected from DNA damaging agents, anti-metabolites, anti-tumour antibiotics, natural products and their analogues, dihydrofolate reductase inhibitors, pyrimidine analogues, purine analogues, cyclin-dependent kinase inhibitors, thymidylate synthase inhibitors, DNA intercalators, DNA cleavers, topoisomerase inhibitors, anthracyclines, vinca drugs, mitomycins, bleomycins, cytotoxic nucleosides, pteridine drugs, diynenes, podophyllotoxins, platinum containing drugs, differentiation inducers and taxanes.

Even more preferably, the drug moiety is selected from methotrexate, methopterin, dichloromethotrexate, 5-fluorouracil, 6-mercaptopurine, tri-substituted purines such as olomoucine, roscovitine and bohemine, flavopiridol, staurosporin, cytosine arabinoside, melphalan, leurosine, actinomycin, daunorubicin, doxorubicin, mitomycin D, mitomycin A, caminomycin, aminopterin, tallysomycin, podophyllotoxin (and derivatives thereof), etoposide, cisplatinum, carboplatinum, vinblastine, vincristine, vindesin, paclitaxel, docetaxel, taxotere retinoic acid, butyric acid, acetyl spermidine, tamoxifen, irinotecan and camptothecin.

In one preferred embodiment, the drug moiety is directly linked to the carrier moiety.

In another preferred embodiment, the drug moiety is indirectly linked to the carrier moiety by means of a linker moiety.

In another preferred embodiment, each carrier moiety bears more than one drug moiety.

In one preferred embodiment, where each carrier moiety bears more than one drug moiety, the drug moieties are different.

In one preferred embodiment, where each carrier moiety bears more than one drug moiety, each drug moiety is linked to the carrier moiety by way of a linker moiety. In one particularly preferred embodiment, each drug moiety is linked to the carrier moiety by an identical linker moiety. In an alternative embodiment, each drug moiety is linked to the carrier moiety by a different linker moiety.

In a further preferred embodiment of the invention, the delivery system may further comprise a targeting moiety. The targeting moiety is capable of directing the delivery system to the specific cell type to which it is preferable for the drug moiety to function. Thus, the targeting moiety acts as an address system biasing the bodies natural distribution of drugs or the delivery system to a particular cell type. The targeting moiety may be attached to the drug moiety or alternatively to the carrier moiety.

In one preferred embodiment, the targetting moiety is directly linked to the carrier moiety.

In another preferred embodiment, the targetting moiety is indirectly linked to the carrier moiety by means of a linker moiety.

Direct linkage may occur through any convenient functional group on the targetting moiety, such as a hydroxy, carboxy or amino group. Indirect linkage will occur through a linking moiety. Suitable linking moieties include bi- and multi-functional alkyl, aryl, aralkyl or peptidic moieties, alkyl, aryl or aralkyl aldehydes acids esters and anhydrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides and the like. The functional groups on the linker moiety used to form covalent bonds to the targetting moiety may be two or more of, e.g., amino, hydrazino, hydroxyl, thiol, maleimido, carbonyl, and carboxyl groups, etc. The linker moiety may include a short sequence of from 1 to 4 amino acid residues that optionally includes a cysteine residue through which the linker moiety bonds to the targetting moiety. Alternatively, the targetting moiety may be linked by leucine zippers, dimerisation domains, or an avidin/biotin linker.

### Process for Preparing Conjugate

A further aspect of the invention relates to a process for preparing a conjugate, said process comprising reacting a compound of formula Ic, or a pharmaceutically acceptable salt thereof,

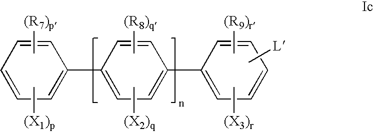

wherein
X₁, X₂ and X₃ are each independently

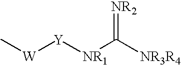

where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
   R₇, R₈ and R₉ are each independently selected from H, alkyl, halo, CF₃, OH, alkoxy, NH₂, CN, NO₂ and COOH;
   p, q and r are each independently 1, 2, 3 or 4;
   q and r are each independently 1, 2, 3 or 4;
   q′ and r′ are each independently 0, 1, 2 or 3, where q+q′ and r+r′ each equal 4;
   p is 1, 2, 3, 4 or 5, and p′ is 0, 1, 2, 3 or 4, where p+p′ is 5;
   L′ is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH,

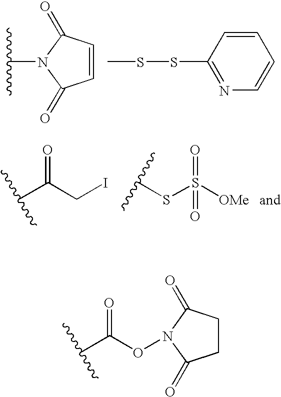

with a cargo moiety selected from a protein, a peptide, an antibody and a drug.

### Process for Preparing Compounds of Formula I

Another aspect of the invention relates to a process for preparing a compound of formula I as defined above, said process comprising the steps of:
(i) reacting a compound of formula II with TsOCH₂CH₂NHBoc to form a compound of formula III, where L′″ is (Z)ₘNR₅R₆ and R₅ and R₆ are an NH₂ protecting group;

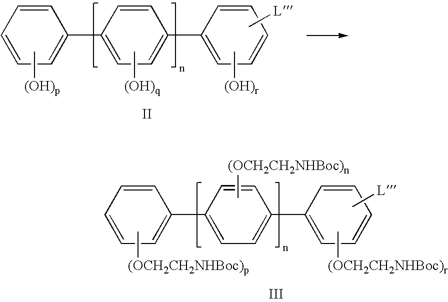

(ii) removing the Boc groups from said compound of formula III to form a compound of formula IV; and
(iii) reacting said compound of formula IV with a compound of formula V to form a compound of formula I.

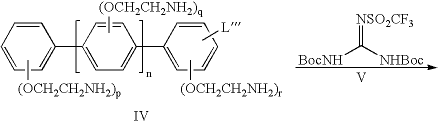

For ease of reference, substituents R₇, R₈ and R₉ have been omitted from the above chemical representations of intermediates II, III and IV.

Alternatively, said compound of formula I may be prepared by a process comprising the steps of:
(i) reacting a compound of formula II with ClCH₂CN to form a compound of formula VI;

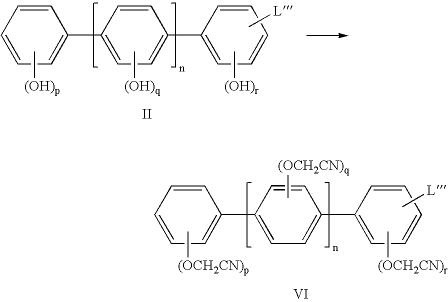

(ii) reacting said compound of formula VI with Pd/H₂/carbon to form a compound of formula IV; and

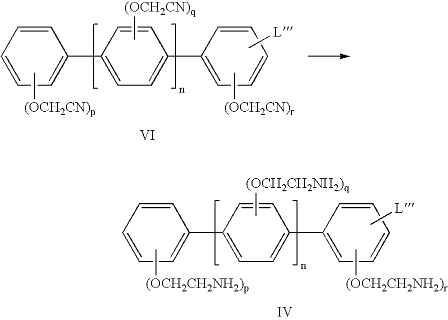

(iii) reacting said compound of formula IV with a compound of formula V to form a compound of formula I.

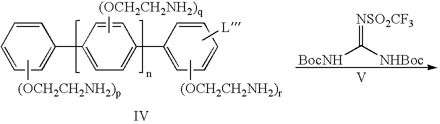

Preferably, said compound of formula II is prepared by the steps of:
(i) reacting a compound of formula VI with a compound of formula VII,

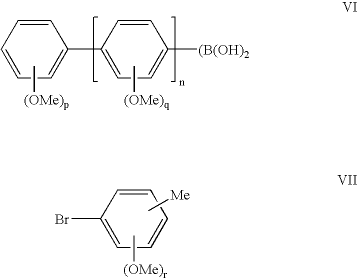

(ii) converting the product obtained from step (i) to a compound of formula II.

Preferably, the reaction between said compound of formula VI and said compound of formula VII is carried out in the presence of a palladium catalyst, more preferably Pd(PPh₃)₄.

The above process steps are equally applicable to the preparation of compounds of formula If.

### Pharmaceutical Compositions

Another aspect of the invention relates to a pharmaceutical composition comprising a compound or conjugate as defined above admixed with one or more pharmaceutically acceptable diluents, excipients or carriers. Even though the compounds and conjugates of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

### Salts/Esters

The compounds of the invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers and tautomers of compounds of formula I. The man skilled in the art will recognise compounds that possess optical properties (one or more chiral carbon atoms) or tautomeric characteristics. The corresponding enantiomers and/or tautomers may be isolated/prepared by methods known in the art.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

The present invention also includes all suitable isotopic variations of the compound or pharmaceutically acceptable salt thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Solvates

The present invention also includes the use of solvate forms of the compounds of the present invention. The terms used in the claims encompass these forms.

### Polymorphs

The invention furthermore relates to the compounds and/or conjugates of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

The invention further includes the compounds of the present invention in prodrug form. Such prodrugs are generally compounds of formula I wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight. In an exemplary embodiment, one or more doses of 10 to 150 mg/day will be administered to the patient.

### Combinations

In a particularly preferred embodiment, the one or more compounds and/or conjugates of the invention are administered in combination with one or more other therapeutically active agents, for example, existing drugs available on the market. In such cases, the compounds of the invention may be administered consecutively, simultaneously or sequentially with the one or more other therapeutically active agents.

Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance in cells which would have been otherwise responsive to initial chemotherapy with a single agent.

By way of example, numerous combinations are used in current treatments of cancer and leukemia. A more extensive review of medical practices may be found in “Oncologic Therapies” edited by E. E. Vokes and H. M. Golomb, published by Springer.

By way of summary, the present invention has demonstrated that designed SMCs are efficient transporters of small molecules across the cell membrane. The SMCs described herein, and derivatives thereof, have many potential applications for in vitro biology, particularly in the transport of peptides, proteins and oligonucleotides into cells. The direct transport of proteins into cells would transform many aspects of molecular and cell biology, as SMC-protein transduction bypasses cellular transcriptional and translational regulatory mechanisms that transfected DNA relies upon. Using SMCs, the effects of proteins in cells can be directly assessed. The technology also facilitates the elucidation of effective physiological concentrations, as the amounts of SMC-protein applied can be finely controlled. For novel drug discovery, the ease of production and scalability of SMCs allows high throughput screening of SMC-peptide or SMC-oligonucleotide libraries for their biological activity in various functional assays. These membrane tugs also have in vivo applications in enhancing transport of proteins, peptides and small molecules to specific areas or around the body. As a cancer therapeutic, for example, approximately 70% of tumours are deficient in p53. Introducing active p53 protein back into these tumors using SMCs will enable them to arrest their growth and activate the programmed cell death pathways that normally restrain cancers. SMCs also have applications in vaccine development as a tool to deliver immunoreactive antigens from pathogens, such as tuberculosis or human papillomavirus. The broad range of possible uses for SMCs demands further investigation, as SMCs have the potential to overcome many of the difficulties that scientists encounter during their research and represents an entirely new methodology to be exploited for drug discovery and clinical applications.

The present invention is further described by way of example, and with reference to the following figures wherein:

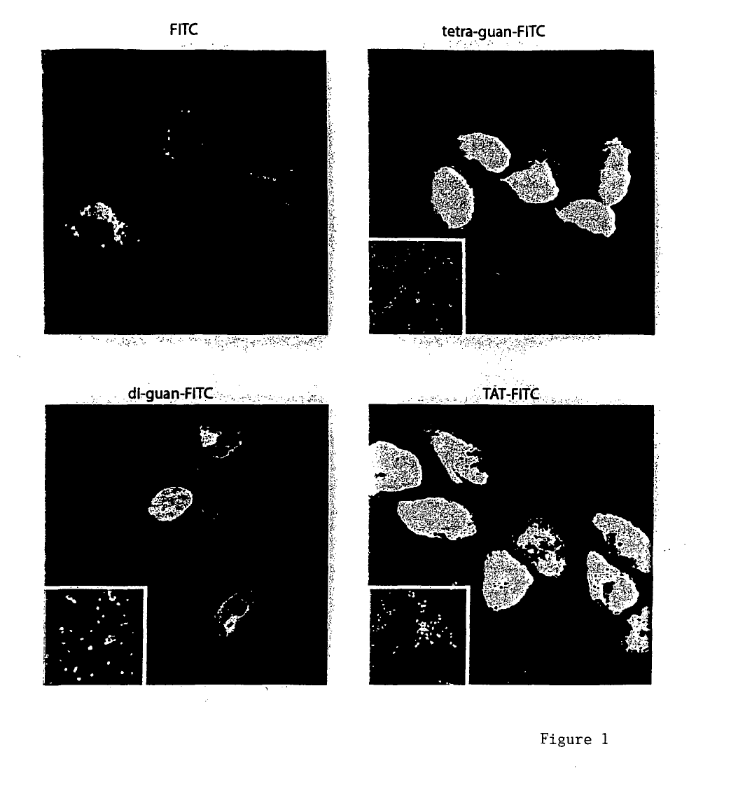

FIG. 1 shows uptake by human osteosarcoma cells (U2OS) of FITC coupled to a di-guanidine carrier according to the invention (compound 13), FITC coupled to a tetra-guanidine carrier according to the invention (compound 26), compared to FITC alone, and FITC coupled to TAT. Uptake was visualised using confocal microscopy.

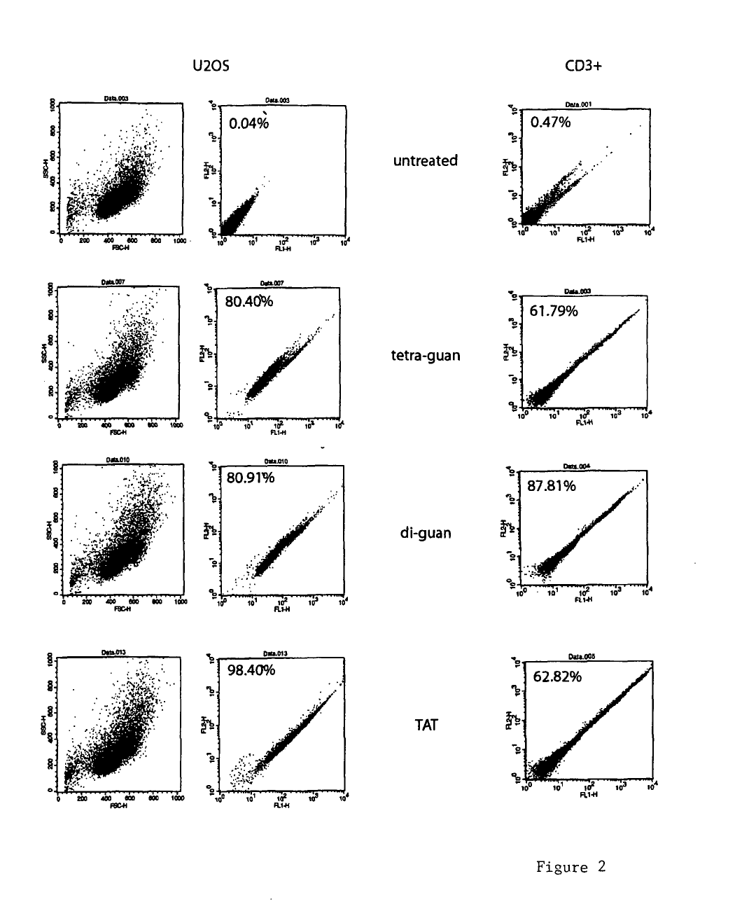

FIG. 2 (left two columns) shows FACS analysis of U2OS cells treated with FITC coupled to a di-guanidine carrier according to the invention (compound 13), FITC coupled to a tetra-guanidine carrier according to the invention (compound 26), FITC alone, and FITC coupled to TAT. FIG. 2 (right column) shows FACS analysis of primary human cells (CD3+ cells).

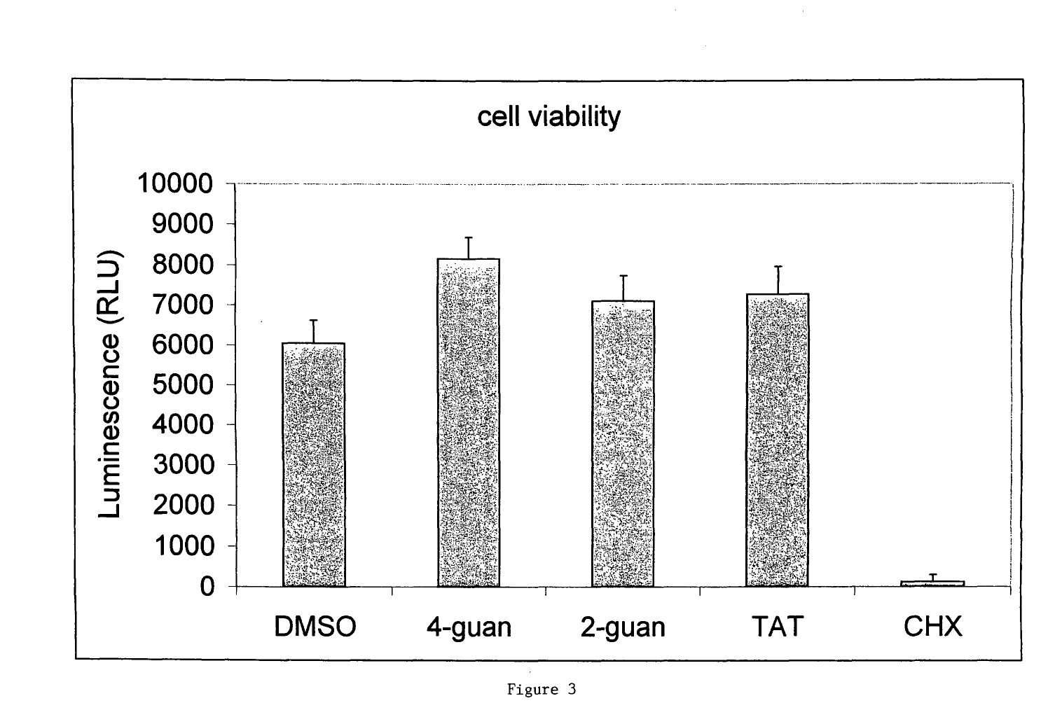

FIG. 3 shows the results of a cell viability experiment in U2OS cells treated with compound 13, compound 26, and FITC coupled to TAT compared to DMSO vector alone and cells treated with cycloheximide (CHX).

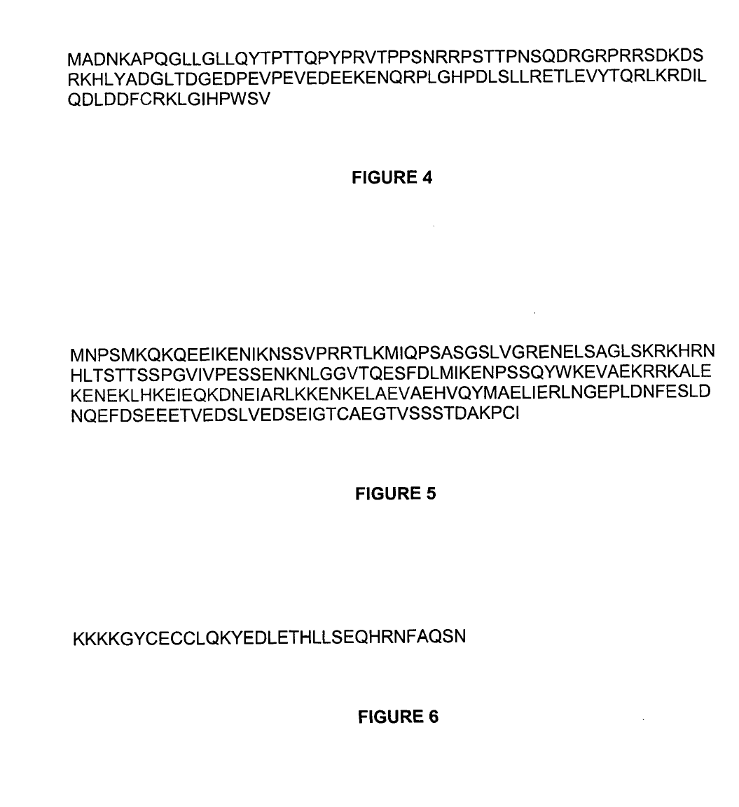

FIG. 4 shows the amino acid sequence of HPV1 E1̂E4 peptide (125 amino acids).

FIG. 5 shows the amino acid sequence of full length Geminin (209 amino acids).

FIG. 6 shows the amino acid sequence of a small peptide based on the Dbf4/ASK regulator for Cdc7 kinase activity.

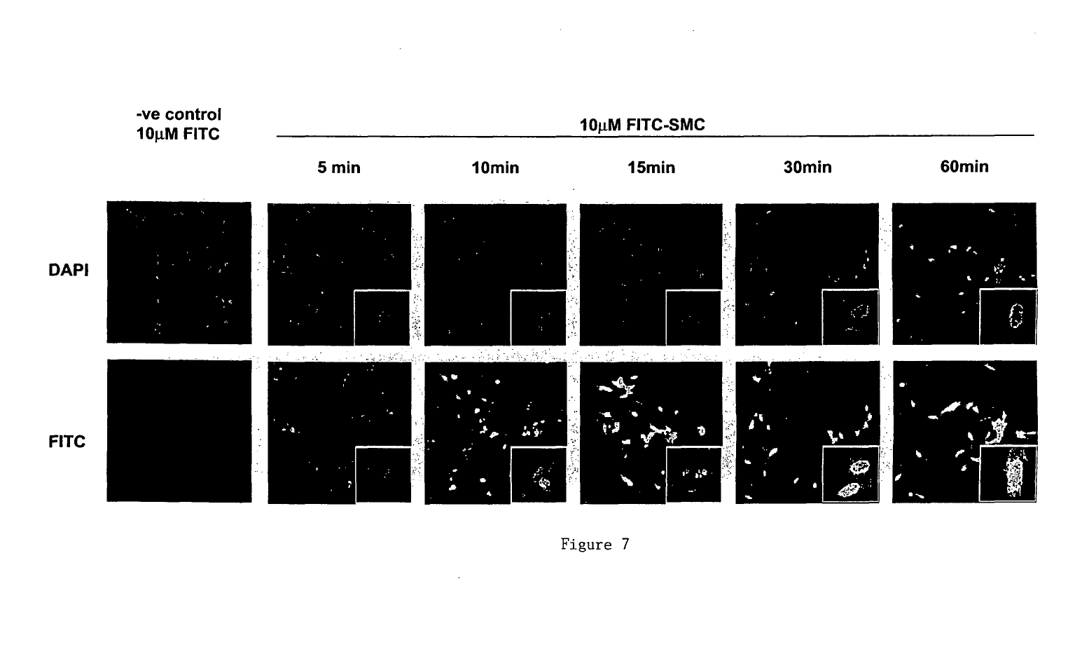

FIG. 7 shows NIH3T3 fibroblasts grown on coverslips were incubated with 10 μM FITC-SMC for the indicated time periods and 10 μM unconjugated FITC as a control. Cells were counterstained with DAPI.

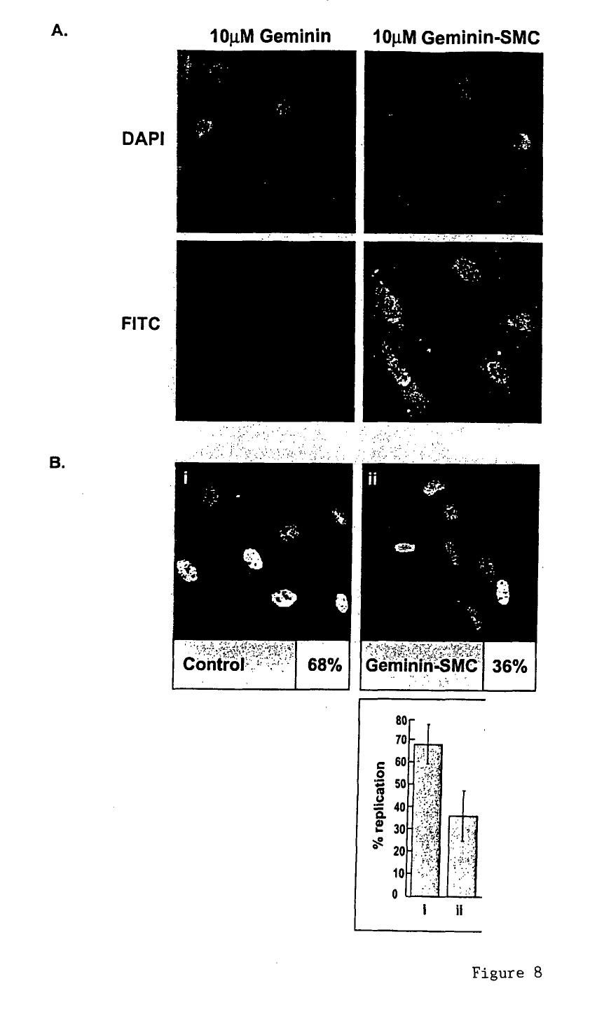

FIG. 8 shows:

A) NIH3T3 fibroblasts were incubated for one hour with 10 μM recombinant His6-Geminin or 10 μM Geminin-SMC conjugate. Cells were fixed, permeabilised and stained with a polyclonal rabbit anti-Geminin primary, a FITC-conjugated anti-rabbit secondary and counterstained with DAPI. In the presence of Geminin-SMC strong FITC staining can be seen within the cells, whilst with the unconjugated form of Geminin no FITC staining can be seen.
B) NIH3T3 fibroblasts were driven into quiescence by density-dependent growth arrest and after 5 days were released back into the cell cycle by subculturing into fresh growth medium. 8 hours after release from quiescence 10 μM of Geminin-SMC was added to the cells. At 21 hours following release cells were pulse-labelled for one hour with 50 μM BrdU. Subsequently cells were fixed, permeabilised and stained with FITC-conjugated anti-BrdU and counterstained with propidium iodide. In a control population 68% of cells were able to re-enter the cell cycle following release whilst in the presence of Geminin-SMC replication dropped by 47% respectively.

### EXAMPLES

### Example 1

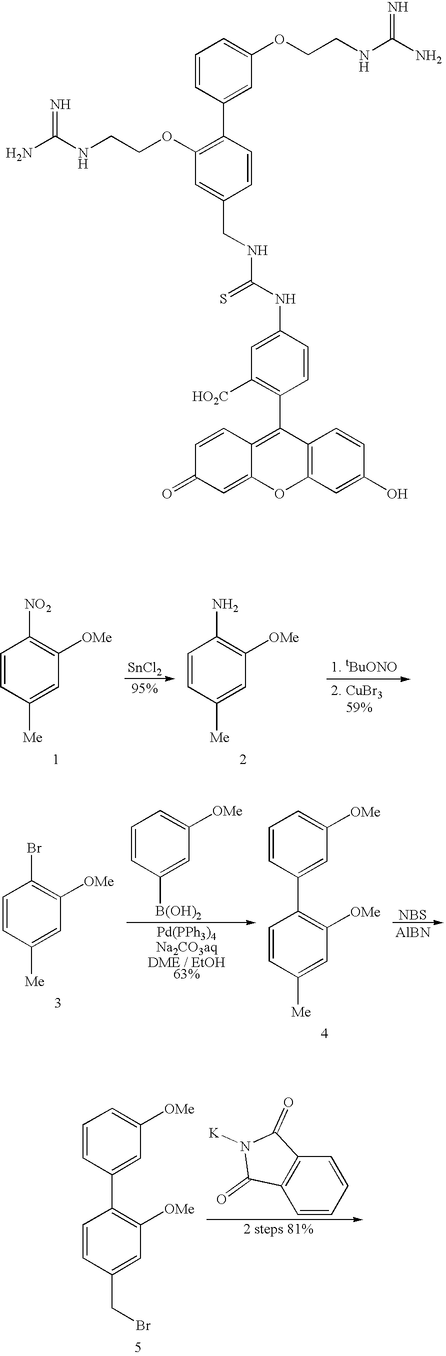

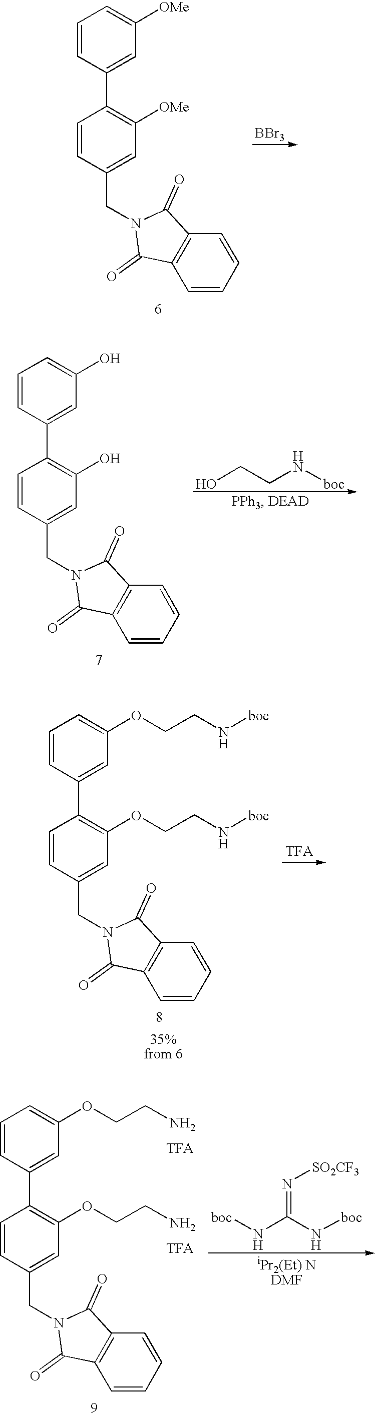

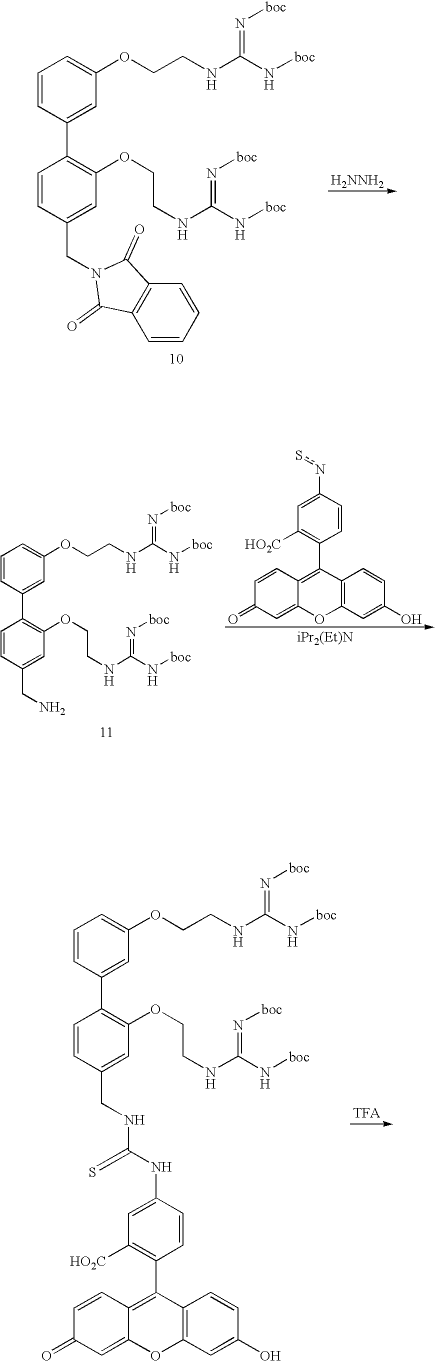

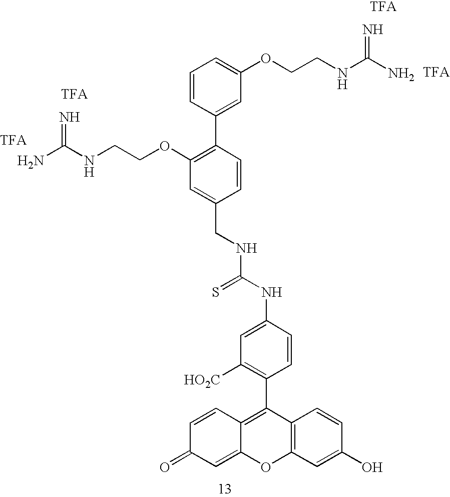

### 2-Methoxy-4-methyl-phenylamine (2)

A solution of substituted nitrobenzene 1 (3.00 g, 17.9 mmol) and SnCl₂.2H₂O (20.2 g, 89.5 mmol) in MeOH (120 ml) was refluxed for 3 h. The mixture was concentrated under vacuum and taken into AcOEt and satd. NaHCO₃aq The organic layer was washed with satd. NaHCO₃aq×3, dried over Na₂SO₄ to afford 2 (2.37 g, 95% yield).

¹H-NMR (CDCl₃): δ 2.27 (s, 3H), 3.84 (s, 3H), 6.56-6.66 (m, 3H). ¹³C-NMR (CDCl₃): δ 21.0, 55.4, 111.5, 115.1, 121.2, 128.1, 133.5, 147.4.

### Bromo-2-methoxy-4-methyl-benzene (3)

Ref; Bambal, R.; Hanzlik, R. P. J. Org. Chem. 1994, 59, 729.

Cupric bromide (4.88 g, 21.8 mmol) in acetonitril (20 ml) was treated with tert-Butylnitrite (2.13 ml, 17.9 mmol) at rt and warmed to 65° C. under nitrogen. A solution of 2 (2.24 g, 16.3 mmol) in acetonitril (20 ml) was added slowly and stirred for additional 15 min. The solvent was removed under vacuum and taken into cyclo-hexane, washed with aqueous NH₃×3, water and dried over Na₂SO₄. The solvent was removed under vacuum and purified by flash chromatography (chloroform) to afford 3 (1.92 g, 59% yield).

¹H-NMR (CDCl₃): δ 2.32 (s, 3H), 3.86 (s, 3H), 6.64 (dd, J=8.0, 1.4 Hz, 1H), 6.72 (d, J=1.4 Hz, 1H), 7.39 (d, J=8.0 Hz, 1H). ¹³C-NMR (CDCl₃): δ 21.4, 56.1, 108.3, 113.1, 122.5, 132.9, 138.7, 155.6.

### 2,3′-Dimethoxy-4-methyl-biphenyl (4)

Ref; Yonezawa, S.; Komurasaki, T.; Kawada, K.; Tsuri, T.; Fuji, M.; Kugimiya, A.; Haga, N.; Mitsumori, S.; Inagaki, M.; Nakatani, T.; Tamura, Y.; Takechi, S.; Taishi, T.; Ohtani, M. J. Org. Chem. 1998, 63, 5831.

To a solution of 3 (610 mg, 3.03 mmol) in DME (12 ml) and EtOH (3 ml) were added 3-methoxy-phenylboronic acid (553 mg, 3.64 mol), 2 M Na₂CO₃ solution (6 ml) and Pd(PPh₃)₄ (176 mg, 0.152 mmol), and refluxed for 17 h under nitrogen. After cooling to rt, the mixture was diluted with hexane/AcOEt (1/1) and washed with water×3, brine, and dried over Na₂SO₄. The solvent was removed under vacuum and purified by flash chromatography (hexane/dichloromethane=2/1) to afford 4 (529 mg, 76% yield).

¹H-NMR (CDCl₃): δ 2.43 (s, 3H), 3.82 (s, 3H), 3.86 (s, 3H), 6.83-6.91 (m, 3H), 7.11-7.15 (m, 2H), 7.25 (d, J=7.6 Hz, 1H), 7.34 (t, J=7.6 Hz, 1H). ¹³C-NMR (CDCl₃): δ 21.6, 55.3, 55.6, 112.3, 115.4, 121.5, 122.1, 127.8, 128.9, 130.6, 138.8, 140.0, 156.4, 159.3.

### 2-(2,3′-Dimethoxy-biphenyl-4-ylmethyl)-isoindole-1,3-dione (6)

To a solution of 4 (529 mg, 2.32 mmol) in CCl4 (48 ml) were added NBS (392 mg, 2.20 mmol) and AIBN (34 mg). After refluxed for 2.5 h, the mixture was cooled to 0° C., filtered. The solvent was removed under vacuum to afford crude 5, which was used without further purification.

A solution of crude 5 and potassium phthalimide (430 mg, 2.32 mmol) in DMF (7.5 ml) was stirred at 80° C. for 1.5 h. After cooling to rt, the mixture was diluted with hexane/AcOEt (1/1) and washed with water×4, brine, and dried over Na₂SO₄. The solvent was removed under vacuum and purified by flash chromatography (hexane/AcOEt=3/1) to afford 6 (574 mg, 66% yield).

¹H-NMR (CDCl₃): δ 3.81 (s, 6H), 4.87 (s, 2H), 6.86 (dd, J=8.0, 2.4 Hz, 1H), 7.02-7.10 (m, 4H), 7.24-7.29 (m, 2H), 7.71 (dd, J=5.4, 3.0 Hz, 2H), 7.86 (dd, J=5.4, 3.0 Hz, 2H). ¹³C-NMR (CDCl₃): δ 41.6, 55.2, 55.7, 111.8, 112.6, 115.2, 121.0, 122.0, 123.4, 128.9, 130.2, 131.0, 132.2, 134.0, 137.0, 139.5, 156.6, 159.2, 168.1.

### 2-[2,3′-bis(2-tert-butyloxycarbonylamino-ethyloxy)-biphenyl-4-ylmethyl]-isoindole-1,3-dione (8)

The dimethoxy derivative 6 (72 mg, 1.9 mmol) was dissolved in dichloromethane (5 ml) and treated with 1.0 M dichloromethane solution of BBr₃ (1.0 ml) at 0° C. and allowed to warm to rt. After stirring over night, the reaction mixture was cooled to 0° C. and treated with 3 ml of MeOH, and then, removed the solvent under vacuum. The residue was taken into AcOEt and washed with 1N HCl×2, water and brine, dried over Na₂SO₄. The solvent was removed under vacuum and roughly purified by flash chromatography (hexane/AcOEt=1/1) to afford 7 (61 mg).

To a solution of 7 (56 mg, 0.16 mmol), 2-tert-butyloxycarbonylamino-ethanol (65 mg, 0.41 mmol) and triphenylphosphine (106 mg, 0.41 mmol) in THF (2 ml) was added DEAD (71 mg, 0.41 mmol) at rt, and stirred for 24 h. The mixture was diluted with AcOEt and washed with 1M Na₂CO₃×2, dried over Na₂SO₄. The solvent was removed under vacuum and purified by flash chromatography (hexane/AcOEt=2/1) to afford 8 (36 mg, 35% yield).

¹H-NMR (CDCl₃): δ 1.41 (s, 9H), 1.43 (s, 9H), 3.41 (m, 2H), 3.52 (m, 2H), 3.99-4.05 (m, 4H), 4.73 (br s, 1H), 4.85 (s, 2H), 5.02 (br s, 1H), 6.85 (dd, J=8.0, 2.4 Hz, 1H), 7.00-7.12 (m, 4H), 7.24-7.32 (m, 2H), 7.72 (dd, J=5.4, 3.0 Hz, 2H), 7.86 (dd, J=5.4, 3.0 Hz, 2H) ¹³C-NMR (CDCl₃): δ 28.40, 40.0, 40.1, 41.5, 67.2, 68.2, 113.0, 113.6, 115.7, 121.7, 122.3, 123.5, 129.0, 130.5, 131.0, 132.1, 134.1, 137.2, 139.5, 155.5, 155.8, 155.9, 168.1.

### 2-{2,3′-bis[2-(N,N′-bis-Boc-guanidino)-ethyloxy]-biphenyl-4-ylmethyl}-isoindole-1,3-dione (10)

Ref; Feichtinger, K; Sings, H. L.; Baker, T. J.; Matthews, K.; Goodman, M. J. Org. Chem. 1998, 63, 8432; Feichtinger, K; Zapf, C.; Sings, H. L.; Goodman, M. J. Org. Chem. 1998, 63, 3804.

Compound 8 (36 mg, 0.057 mmol) was dissolved in TFA (2 ml) and stirred at rt for 2.5 h. The solvent was removed under vacuum to give 9, which was used without further purification.

To a solution of 9 in DMF (0.8 ml) were added N,N-di-Boc-N′-trifluoromethanesulfonyl-guanidine (67 mg, 0.17 mmol) and i-Pr₂(Et)N (60 μl, 0.34 mmol) and stirred at rt for 18 h. The reaction mixture was diluted with AcOEt and washed with 1N HCl×3, water and brine, dried over Na₂SO₄. The solvent was removed under vacuum and purified by preparative TLC (hexane/AcOEt=1/1) to afford 10 (47 mg, 90% yield).

¹H-NMR (CDCl₃): δ 1.47 (s, 18H), 1.49 (s, 18H), 3.74-3.86 (m, 4H), 4.04-4.10 (m, 4H), 4.85 (s, 2H), 6.87 (dd, J=8.0, 2.0 Hz, 1H), 6.97 (br s, 1H), 7.04 (s, 1H), 7.09 (d, J=7.7 Hz, 1H), 7.21 (d, J=7.7 Hz, 1H), 7.26-7.31 (m, 2H), 7.71 (dd, J=5.4, 3.0 Hz, 2H), 7.86 (dd, J=5.4, 3.0 Hz, 2H), 8.62 (br t, J=5.6 Hz, 1H), 8.74 (br t, J=5.1 Hz, 1H) ¹³C-NMR (CDCl₃): δ 28.0, 28.3, 40.1, 40.3, 41.5, 66.3, 66.9, 112.8, 113.3, 115.8, 121.5, 123.0, 123.5, 128.9, 130.3, 131.2, 132.1, 134.1, 137.0, 139.3, 152.9, 153.0, 155.4, 156.38, 156.43, 158.4, 163.30, 163.33, 168.1.

### 2-[2,3-bis(2-guanidino-ethyloxy)-biphenyl-4-ylmethyl]-thioureido-fluorescein (13)

To a solution of 10 (22 mg, 0.024 mmol) in EtOH (0.6 ml) was added hydrazine monohydrate (12 μl, 0.24 mmol) and stirred at rt for 18 h. The solvent was evaporated under vacuum. The residue was taken into dichloromethane and the precipitate was filtered off. The filtrate was washed with brine, and the water layer was extracted with dichloromethane×3. The combined organic layer was dried over Na₂SO₄. The solvent was removed under vacuum to afford crude 11, which was used without further purification.

Compound 11 was dissolved in DMF (1.0 ml) and fluorescein isothiocyanate isomer I (19 mg, 0.048 mmol) and i-Pr₂(Et)N (17 μl, 0.096 mmol) and stirred at rt in a dark for 20 h. The reaction mixture was diluted with AcOEt and washed with 1N HCl×3, water and brine, dried over Na₂SO₄. After remove the solvent, the residue was dissolved in THF (1.5 ml) and treated with PS-trisamine (Argonaut Technologies Inc., 4.17 mmol/g, 20 mg) to remove the remaining FITC. After agitaing at rt for 15 min, the resin was filtered off and the solvent was removed to give crude 12, which was dissolved in TFA (1.5 ml) and stirred at rt for 2 h. TFA was removed under vacuum, the residue was purified by reverse-phase HPLC using a preparative C-18 column (0.1% TFA H₂O/acetonitril) to afford 13 (8 mg, 30% yield).

¹H-NMR (MeOH-d4): δ 3.54 (m, 2H), 3.61 (m, 2H), 4.15 (m, 4H), 4.91 (s, 2H), 6.64 (d, J=9.0 Hz, 2H), 6.78-6.83 (m, 4H), 6.93 (d, J=7.9 Hz, 1H), 7.05-7.35 (m, 9H), 7.81 (d, J=8.2 Hz, 1H), 8.22 (s, 1H) MS (MALDI) m/z 774 (calcd), 775 (M+1, found).

### Example 2

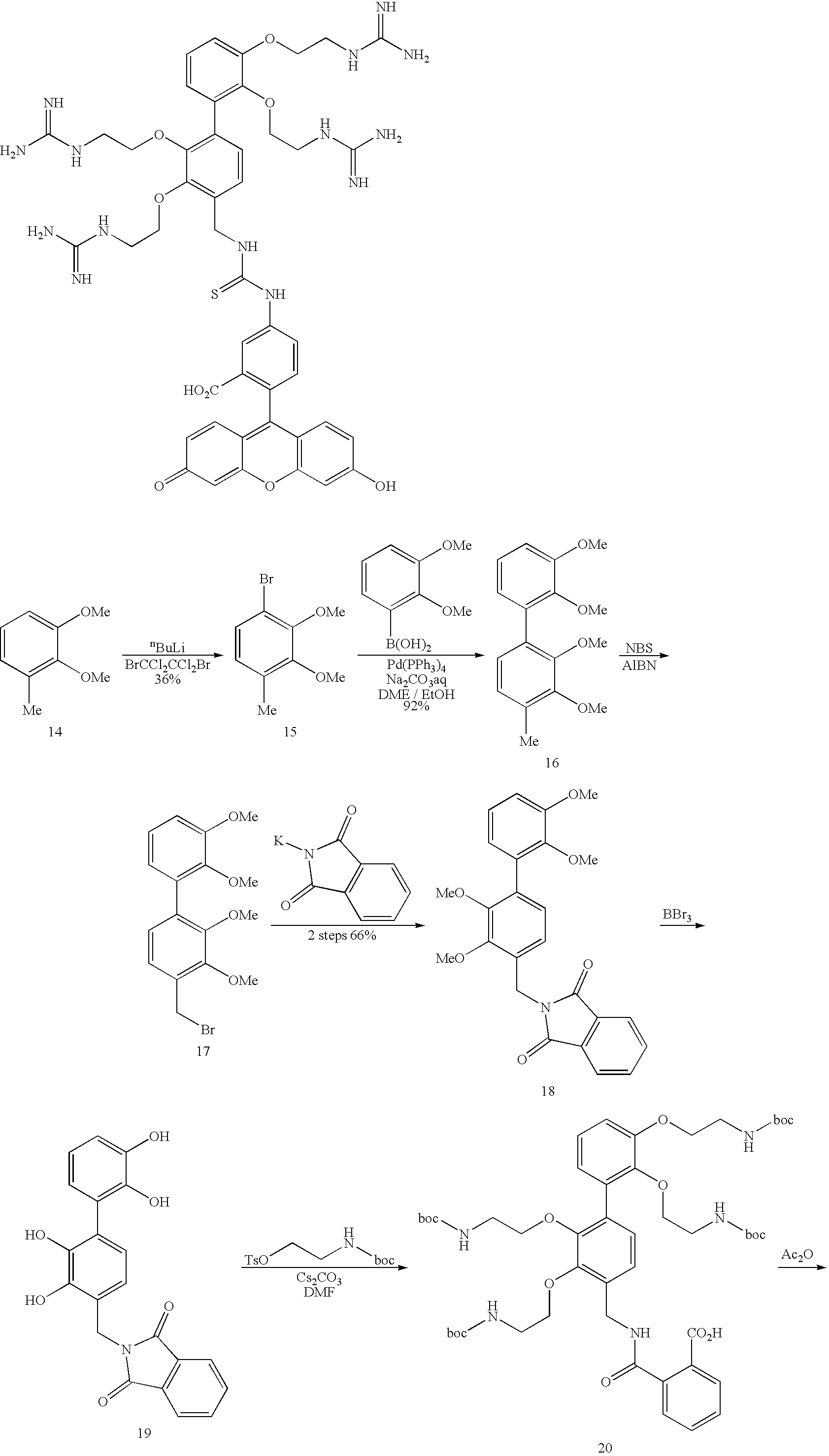

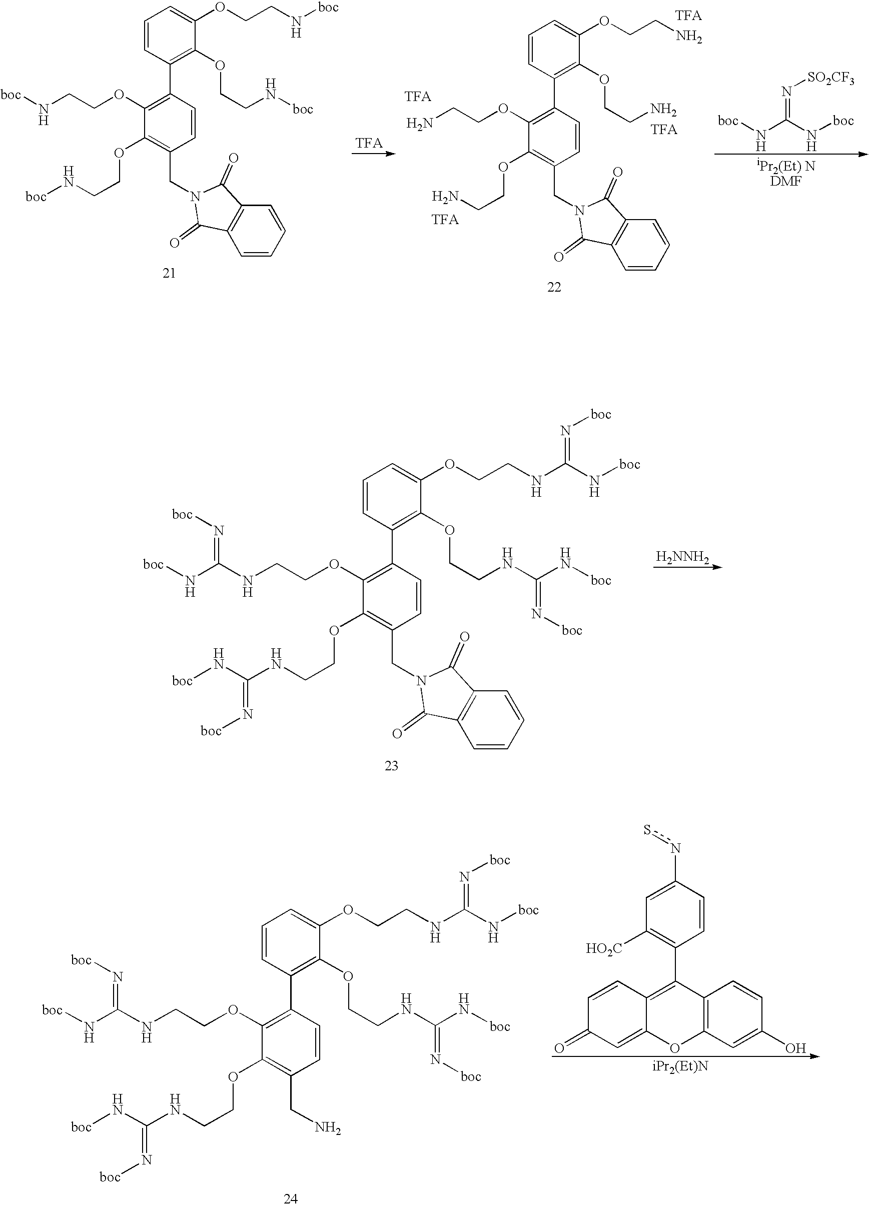

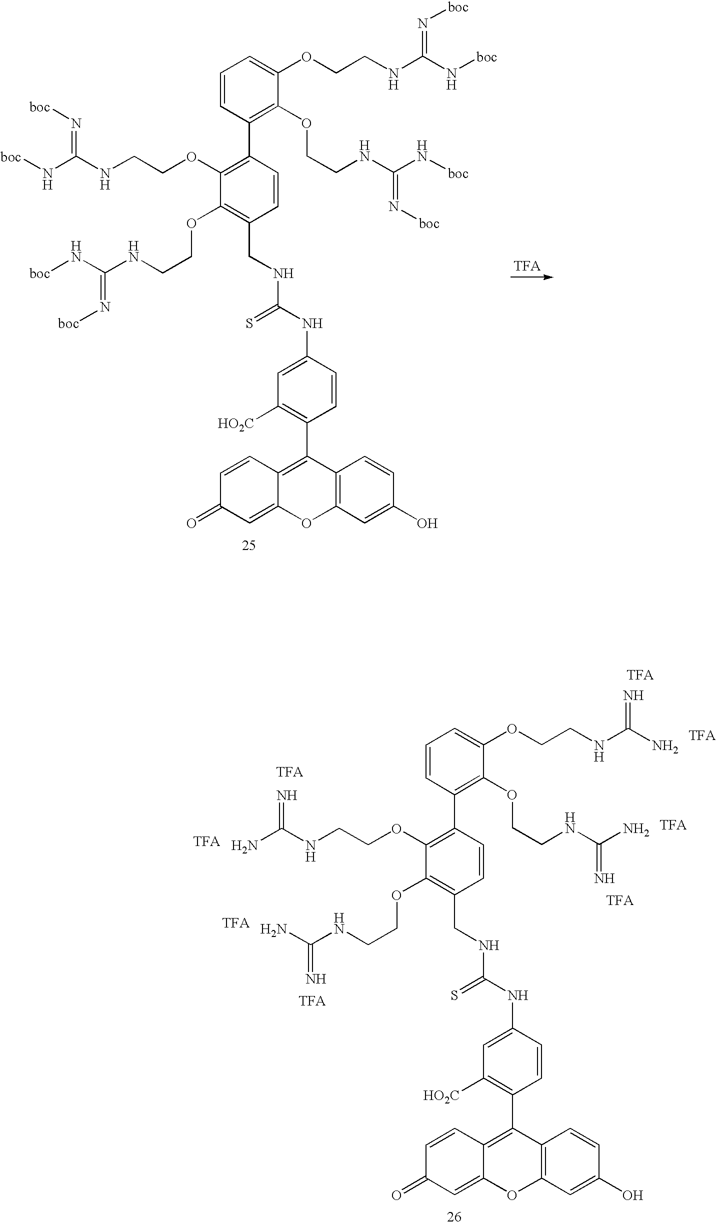

### 1-Bromo-2,3-dimethoxy-4-methyl-benzene (15)

Ref; Esteban, G; López-Sánchez, M. A.; Martinez, M. E.; Plumet, J. Tetrahedron 1998, 54, 197; Bringmann, G; Günther, C.; Peters, E.; Peters, K. Tetrahedron 2001, 57, 1253.

n-BuLi 1.6M in hexane (18.5 ml, 29.6 mmol) was added dropwise at 0° C. to a solution of 2,3-dimethoxytoluene (3.00 g, 19.7 mmol) and TMEDA (2.97 ml, 19.7 mmol) in anhydrous ether (50 ml) under nitrogen. After stirring at rt for 2 h, the reaction mixture was cooled to −78° C. and (CBrCl₂)₂ (9.64 g, 29.6 mmol) was added. After stirring for 10 min, the bath was taken off and allowed to warm to rt. The reaction mixture was diluted with ether and washed with water, 1N HCl×2 and brine, dried over Na₂SO₄. The solvent was removed under vacuum and purified by flash chromatography (hexane/dichloromethane=5/1) to afford 15 (1.62 g, 36% yield).

¹H-NMR (CDCl₃): δ 2.22 (s, 3H), 3.85 (s, 3H), 3.88 (s, 3H), 6.79 (d, J=8.3 Hz, 1H), 7.16 (d, J=8.3 Hz, 1H). ¹³C-NMR (CDCl₃): δ 15.7, 60.4, 60.6, 114.6, 126.7, 127.4, 132.1, 150.4, 152.5.

### 2,3,2′,3′-Tetramethoxy-4-methyl-biphenyl (16)

To a solution of 15 (500 mg, 2.16 mmol) in DME (8.7 ml) and EtOH (2.2 ml) were added 2,3-dimethoxy-phenylboronic acid (472 mg, 2.59 mmol), 2 M Na₂CO₃ solution (4.3 ml) and Pd(PPh₃)₄ (125 mg, 0.108 mmol), and refluxed for 18.5 h under nitrogen. After cooling to rt, the mixture was diluted with hexane/AcOEt (1/1) and washed with water×3, brine, and dried over Na₂SO₄. The solvent was removed under vacuum and purified by flash chromatography (dichloromethane) to afford 16 (573 mg, 92% yield).

¹H-NMR (CDCl₃): δ 2.31 (s, 3H), 3.63 (s, 3H), 3.67 (s, 3H), 3.87 (s, 3H), 3.90 (s, 3H), 6.85 (dd, J=7.6, 1.3 Hz, 1H), 6.91-6.94 (m, 3H), 7.07 (t, J=7.9 Hz, 1H). ¹³C-NMR (CDCl₃): δ 15.9, 55.8, 60.1, 60.4, 60.6, 111.6, 123.3, 123.4, 125.0, 125.7, 130.8, 131.7, 133.0, 146.9, 150.8, 151.3, 152.8.

### 2-(2,3,2′,3′-Tetramethoxy-biphenyl-4-ylmethyl)-isoindole-1,3-dione (18)

To a solution of 16 (573 mg, 1.99 mmol) in CCl4 (42 ml) were added NBS (336 mg, 1.89 mmol) and AIBN (29 mg). After refluxed for 2 h, the mixture was cooled to 0° C., filtered. The solvent was removed under vacuum to afford crude 17, which was used without further purification.

A solution of crude 17 and potassium phthalimide (369 mg, 1.99 mmol) in DMF (6.5 ml) was stirred at 80° C. for 1.5 h. After cooling to rt, the mixture was diluted with hexane/AcOEt (1/1) and washed with water×5, brine, and dried over Na₂SO₄. The solvent was removed under vacuum and purified by flash chromatography (hexane/AcOEt=3/1) to afford 18 (552 mg, 64% yield).

¹H-NMR (CDCl₃): δ 3.62 (s, 3H), 3.65 (s, 3H), 3.89 (s, 3H), 3.99 (s, 3H), 4.98 (s, 2H), 6.81 (dd, J=7.6, 1.5 Hz, 1H), 6.90-6.93 (m, 2H), 6.98 (d, J=8.0 Hz, 1H), 7.06 (t, J=7.9 Hz, 1H), 7.72 (dd, J=5.4, 3.0 Hz, 2H), 7.86 (dd, J=5.4, 3.0 Hz, 2H). ¹³C-NMR (CDCl₃): δ 36.4, 55.8, 60.3, 60.59, 60.63, 111.8, 122.9, 123.1, 123.36, 123.45, 125.9, 129.5, 132.2, 132.5, 132.7, 134.0, 146.9, 151.2, 151.4, 152.8, 168.2.

### 2-[2,3,2′,3′-Tetra(2-tert-butyloxycarbonylamino-ethyloxy)-biphenyl-4-ylmethyl]-isoindole-1,3-dione (21)

Ref; Lu, T.; Tomezuk, B.; Illig, C. R.; Bone, R.; Soll, R. M. Bioorg. Med. Chem. Lett. 1998, 8, 1595.

The tetramethoxy biphenyl derivative 18 (250 mg, 0.577 mmol) was dissolved in dichloromethane (15 ml) and treated with 1.0 M dichloromethane solution of BBr₃ (6.0 ml) at 0° C. and allowed to warm to rt. After stirring over night, the reaction mixture was cooled to 0° C. and treated with 10 ml of MeOH, and then removed the solvent under vacuum. The residue was taken into AcOEt and washed with 1N HCl×2, water and brine, dried over Na₂SO₄. The solvent was removed under vacuum to afford 19 (248 mg), which was used without further purification.

To a solution of crude 19 (105 mg) in DMF (3.0 ml) were added Cs₂CO₃ (654 mg, 2.00 mmol) and tert-butyl N-(2-tosyloxyethyl)-carbamate (526 mg, 1.67 mmol), and heated to 80° C. under nitrogen. After stirring for 4 h, additional Cs₂CO₃ (654 mg, 2.00 mmol) and tert-butyl N-(2-tosyloxyethyl)-carbamate (526 mg, 1.67 mmol) were added. After stirring further 15 h, the reaction mixture was taken into AcOEt and 1N HCl. The organic layer was washed with 1N HCl×2, water and brine, dried over Na₂SO₄. The solvent was removed under vacuum and purified by preparative TLC (CHCl₃/MeOH=10/1) to afford 20, which was dissolved in DMF (2 ml) and acetic anhydride (0.5 ml) was added. After stirring at 80° C. for 1 h, the mixture was diluted with AcOEt and washed with water×3, saturated Na₂CO₃, brine, dried over Na₂SO₄. The solvent was removed under vacuum and purified by preparative TLC (AcOEt/hexane=1/1) to afford 21 (38 mg, 16% yield from 18).

¹H-NMR (CDCl₃): δ 1.38 (s, 9H), 1.42 (s, 9H), 1.43 (s, 9H), 1.45 (s, 9H), 3.10 (m, 2H), 3.19 (m, 2H), 3.58 (m, 4H), 3.78 (m, 4H), 4.11 (m, 2H), 4.23 (m, 2H), 4.64 (br s, 1H), 4.95 (s, 2H), 5.20 (br s, 1H), 5.44 (br s, 1H), 5.78 (br s, 1H), 6.85 (br d, J=6.7 Hz, 1H), 6.93 (br d, J=7.4 Hz, 1H), 6.98 (d, J=8.0 Hz, 1H), 7.08 (t, J=7.9 Hz, 1H), 7.14 (d, J=8.0 Hz, 1H), 7.73 (dd, J=5.4, 3.0 Hz, 2H), 7.87 (dd, J=5.4, 3.0 Hz, 2H).

### 2-{2,3,2′,3′-Tetra[2-N,N′-bis(tert-butoxycarbonyl)guanidino-ethyloxy]-biphenyl-4-ylmethyl}-isoindole-1,3-dione (23)

Compound 21 (38 mg, 0.040 mmol) was dissolved in TFA (2 ml) and stirred at rt for 2 h. The solvent was removed under vacuum to give 22, which was used without further purification.

To a solution of 22 in DMF (1.0 ml) were added N,N-di-Boc-N′-trifluoromethanesulfonyl-guanidine (94 mg, 0.24 mmol) and i-Pr₂(Et)N (84 μl, 0.48 mmol) and stirred at rt for 15 h. The reaction mixture was diluted with AcOEt and washed with 1N HCl×3, water and brine, dried over Na₂SO₄. The solvent was removed under vacuum and purified by preparative TLC (hexane/AcOEt=3/2) to afford 23 (45 mg, 74% yield).

¹H-NMR (CDCl₃): δ 1.36 (s, 9H), 1.41 (s, 9H), 1.44 (s, 9H), 1.45 (s, 18H), 1.48 (s, 27H), 3.45 (m, 4H), 3.88 (m, 8H), 4.16 (m, 2H), 4.39 (m, 2H), 4.98 (s, 2H), 6.83-7.03 (m, 5H), 7.71 (dd, J=5.4, 3.0 Hz, 2H), 7.86 (dd, J=5.4, 3.0 Hz, 2H), 8.46 (br s, 1H), 8.57 (br s, 1H), 8.79 (br s, 1H), 8.89 (br s, 1H).

### [2,3,2′,3′-Tetra(2-guanidino-ethyloxy)-biphenyl-4-ylmethyl]-thioureido-fluorescein (26)

To a solution of 23 (45 mg, 0.030 mmol) in EtOH (0.7 ml) was added hydrazine monohydrate (15 μl, 0.30 mmol) and stirred at rt for 16 h. The solvent was evaporated under vacuum. The residue was taken into dichloromethane and the precipitate was filtered off. The filtrate was washed with brine, and the water layer was extracted with dichloromethane×3. The combined organic layer was dried over Na₂SO₄. The solvent was removed under vacuum to afford crude 24, which was used without further purification.

Compound 24 was dissolved in DMF (1.0 ml) and fluorescein isothiocyanate isomer I (18 mg, 0.045 mmol) and i-Pr₂(Et)N (16 μl, 0.090 mmol) and stirred at rt in a dark for 15 h. To the reaction mixture was added PS-trisamine (Argonaut Technologies Inc., 4.17 mmol/g, 25 mg) to remove the remaining FITC. After agitaing at rt for 30 min, the resin was filtered off and the filtrate was diluted with AcOEt and washed with 1N HCl×3, water, brine, dried over Na₂SO₄. The solvent was removed under vacuum to give crude 25, which was dissolved in TFA (2.0 ml) and stirred at rt for 2 h. TFA was removed under vacuum, the residue was purified by reverse-phase HPLC using a preparative C-18 column (0.1% TFA H₂O/acetonitril) to afford 26 (9 mg, 20% yield from 23).

¹H-NMR (MeOH-d4): δ 3.34 (m, 4H), 3.69 (m, 4H), 3.94-3.97 (m, 4H), 4.22-4.30 (m, 4H), 4.96 (s, 2H), 6.61 (d, J=8.7 Hz, 2H), 6.76-6.78 (m, 4H), 6.90 (d, J=7.3 Hz, 1H), 7.04-7.24 (m, 5H), 7.79 (d, J=8.1 Hz, 1H), 8.29 (s, 1H). MS (MALDI) m/z 976 (calcd), 977, 978, 979 (found).

### Example 3

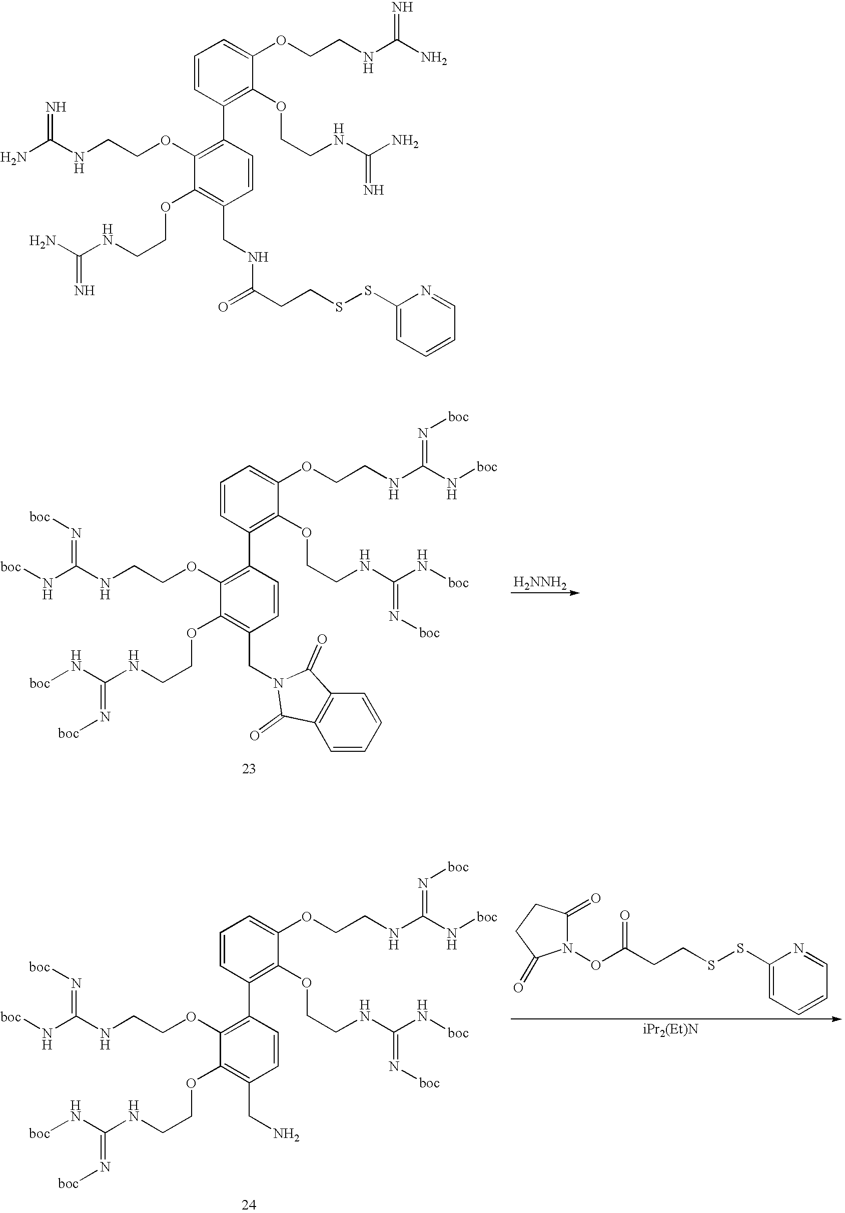

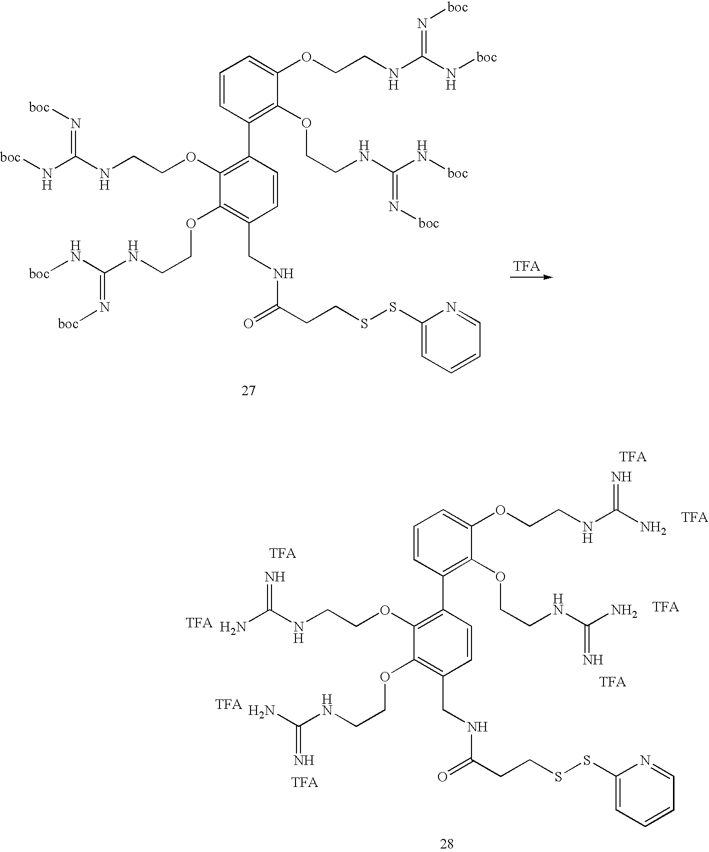

### N-{2,3,2′,3′-Tetra{2-[N,N′-bis(tert-butoxycarbonyl)guanidino]-ethyloxy}-biphenyl-4-ylmethyl}-3-[2-pyridyl)dithio]propionamide (26)

Ref; Cosimelli, B.; Neri, D.; Roncucci, G. Tetrahedron 1996, 34, 11281; Moriarty, R. M.; Liu, K.; Zhuang, H.; Lenz, D.; Brimfield, A.; Xia, C. Synthetic Comm. 1995, 25, 2763.

To a solution of 23 (8 mg, 0.005 mmol) in EtOH (0.5 ml) was added hydrazine monohydrate (3 μl, 0.06 mmol) and stirred at rt for 17 h. The solvent was evaporated under vacuum. The residue was taken into dichloromethane and the precipitate was filtered off. The filtrate was washed with brine, and the water layer was extracted with dichloromethane×3. The combined organic layer was dried over Na₂SO₄. The solvent was removed under vacuum to afford crude 24, which was used without further purification.

To a solution of compound 24 and i-Pr₂(Et)N (2 μl, 0.01 mmol) in dichloromethane (0.3 ml) was added N-succinimidyl-3-(2-pyridyldithio)propionate (3 mg, 0.01 mmol) and stirred at rt in a dark for 24 h. The solvent was removed under vacuum and purified by preparative TLC (EtOAc/hexane=1/1) to afford 27 (4 mg, 50% yield).

¹H-NMR (CDCl₃): δ 1.43-1.51 (m, 72H), 2.70 (t, J=7.2 Hz, 2H), 3.11 (t, J=7.2 Hz, 2H), 3.45-3.50 (m, 4H), 3.84-3.93 (m, 8H), 4.15-4.21 (m, 4H), 4.46 (d, J=5.6 Hz, 2H), 6.89-6.92 (m, 2H), 6.99-7.08 (m, 4H), 7.57-7.68 (m, 2H), 8.36 (m, 1H), 8.46 (br t, 1H), 8.55 (br t, 1H), 8.80 (br t, 2H).

### N-[2,3,2′,3′-Tetra(2-guanidino-ethyloxy)-biphenyl-4-ylmethyl]-3-[2-pyridyl)dithio]propionamide (28)

Compound 27 (4 mg, 0.0024 mmol) was dissolved in TFA (0.8 ml) and stirred at rt for 3 h. TFA was removed under vacuum to afford 28 (4 mg).

¹H-NMR (MeOH-d4): δ 2.73 (t, J=6.6 Hz, 2H), 3.10 (t, J=6.6 Hz, 2H), 3.23-3.30 (m, 4H), 3.62-3.67 (m, 4H), 3.88 (m, 2H), 3.96 (m, 2H), 4.22 (m, 4H), 4.47 (s, 2H), 6.87 (dd, J=7.4, 1.7 Hz, 1H), 7.02 (d, J=7.9 Hz, 1H), 7.08-7.24 (m, 4H), 7.76-7.80 (m, 2H), 8.38 (d, J=4.7 Hz, 1H), 8.70 (br t, 1H). ESMS; m/z calcd; 784, found; 899-[(M+1)+TFA], 785 (M+1), 507 [(M+2)+2TFA], 450 [(M+2)+TFA], 393 (M+2), 338 [(M+3)+2TFA].

### Example 4

### Uptake and Sub-Cellular Localization

In order to observe the ability of the di-guanidine and tetra-guanidine compounds to transport small molecules, they were coupled to FITC, which emits light at 514 nm after laser excitation at 492 nm. Human osteosarcoma cells (U2OS) were cultured on glass coverslips and incubated for 10 minutes with each compound to a final concentration of 10 μM. They were compared to FITC conjugated to TAT and FITC alone. After incubation, cells were rinsed in PBS and mounted, without fixation, onto slides and visualized by confocal microscopy. At lower magnification (10×), cells treated with FITC coupled to either of the guanidine compounds or to TAT appeared strongly fluorescent (inset panels of FIG. 1). Cells were visualized under higher magnification (63×), to assess sub-cellular localization of the FITC. As can be seen in FIG. 1, cells treated with tetra-guanidine-FITC showed intense nuclear fluorescence, while di-guanidine-FITC treatment had both nuclear and cytoplasmic fluorescence. At this level of magnification, nonspecific uptake of fluorescein was detected in small cytoplasmic compartments, which may represent endosomes. These results demonstrate the delivery of FITC to specific sub-cellular compartments of cells by the di- and tetra-guanidine carriers.

### Delivery to Human Adherent Cell Lines and Primary Suspension Cells

The efficiency of delivery of FITC by these guanidine carriers was tested by FACS analysis. U2OS cells were treated with at a concentration of 10 □M with each of the compounds indicated for 15 minutes, and then harvested by trypsinization, and washed in PBS. The live cells were then subjected to FACS analysis. As can be seen in FIG. 2, 80% of the cells treated with either di-guanidine-FITC or tetra-guanidine-FITC were fluorescent above the background auto-fluorescence of U2OS cells. Similar results were also obtained with 293T cells, human embryonic kidney cells transformed with SV40 T antigen. These results are comparable to TAT-FITC, where 98% of cells were fluorescent after treatment.

To determine whether SMCs deliver small molecules to primary human cells, CD3+ cells were isolated by MACS separation from freshly harvested peripheral blood mononuclear cells. This purified cell population was treated with 10 □M of each compound for 15 minutes. Cells were then washed in PBS and analyzed by FACS. 88% of cells treated with di-guanidine-FITC and 62% of cells treated with tetra-guanidine-FITC were fluorescent, as compared to 63% of cells treated with TAT-FITC, indicating efficient delivery of FITC to primary cells by these compounds.

### Cell Viability

The above experiments demonstrate the rapid and efficient delivery of FITC to cells, which occurs within 10-15 minutes of application to cells. However, in order to test the effects of longer exposure to these compounds on viability, cells were incubated for 24 and 48 hours with these compounds, ranging in concentration from 0.1 □M to 10□M concentrations (FIG. 3 and data not shown). 1×10⁴ U2OS cells were seeded and treated as indicated for 24 hours. Cells were then lysed and ATP concentrations were measured as an indicator of cell viability, as dying cells rapidly lose intracellular ATP. As seen in FIG. 3, no decrease in ATP concentration was seen with the SMCs as compared to the DMSO vector alone. Levels of ATP are compared to those for cells treated with cychoheximide (CHX), a protein synthesis inhibitor, which is toxic to cells. These results indicate that the SMC compounds do not decrease the viability of cultured cells.

### Example 5

The SMCs' ability to transfer different classes of biomolecules across biological membranes is demonstrated.

These properties are demonstrated using a functional readout i.e. biological activity of the molecule being delivered. This not only demonstrates delivery/dumping of the cargo to the required location, but furthermore illustrates advantageous retention of function of cargo moities delivered according to the present invention.

The experimental system used to demonstrate these effects is based on the cellular DNA synthesis assay (S-phase assay) described below.

### S-Phase Assay:

Essentially the system is set up as follows: Mouse or human fibroblasts are arrested in quiescence (G0) by contact inhibition.

The quiescent fibroblasts are then subcultured at lower density to release fibroblasts from G0. Re-entry into the DNA synthesis phase (S phase) occurs 21 hours after the release. Pre-replicative complexes (pre-RCs) essential for unwinding of the DNA helix prior to DNA synthesis are assembled at replication origins 16-18 hrs after the release from G0.

One sample of cells is untreated and the other is treated by exposure to an SMC-conjugate according to the present invention. Treatment takes place before pre-RC assembly, preferably at 5-10 hours after release from G0.

The ability of cells to start S phase is monitored with a BrdU pulse at 21 hours. BrdU becomes incorporated into DNA during DNA synthesis and is thus a marker for entry into S phase. BrdU incorporation is then assayed and treated cells are compared with non-treated cells.

### Protocol: S-Phase Assay (Cell Proliferation Assay)

For synchronisation in G0, NIH3T3 fibroblasts are seeded at high density and driven into density-dependent growth arrest. After 5 days cells are re-seeded 1 in 4 on glass coverslips in DMEM supplemented with 10% FCS, 10 U/ml penicillin and 0.1 mg/ml streptomycin.

Samples of cells are then treated at 8 hours, reserving untreated samples for comparison.

DNA synthesis is monitored by pulse labelling cells 21 h after release from G0 for 1 h with 50 μM BrdU. Cells are fixed for 5 min with 4% paraformaldehyde and permeabilised with 0.2% Triton X-100 for 5 min.

Optionally antibody incubations may be performed at this stage to detect particular entities within the cells. For example, cells microinjected with pcDNA3.1E1̂E4 are incubated with anti-E4 MAb 4.37 followed by Texas Red-conjugated goat 14 anti-mouse antibody (Amersham). These cells are then counter-stained with a DNA stain (eg. TOTO 3 (Molecular Probes); see next step).

### Staining:

Cells are re-fixed with 4% paraformaldehyde and incubated with 4M HCl for 1 hour prior to staining with FITC-conjugated mouse anti-BrdU MAb (Alexis).

The percentage of cells undergoing DNA replication is determined from images obtained by confocal fluorescence microscopy on a Leica TCS SP confocal microscope.

### Treatment by Plasmid Microinjection:

DNA plasmids (0.1 ug/ml) expressing HPV1 E1̂E4 (pcDNA3.1E1̂E4), or a fusion protein between the small GTPase ADP-ribosylation factor ARF and CFP (pECFP-N1/ARF) are microinjected into nuclei of synchronised G1 phase cells 8 h after release from G0.

This S-phase assay is sensitive to inhibition of DNA synthesis without being tied to the molecular mechanism of the inhibition (see further examples below). However, in this example, the effects are demonstrated using the HPV1 E1̂E4 protein, for which the molecular mechanism of inhibition is understood:

Background and Role of HPV1 E1̂E4

Sequential assembly of ORC, cdc, cdt1 and minichromosome maintenance proteins (Mcm2-7) into pre-replicative complexes (pre-RCs) at replication origins is essential for initiation of eukaryotic DNA replication.

Mcm7 is a member of a family of six structurally related proteins, Mcm2-7, that are essential replication initiation factors evolutionarily conserved in all eukaryotes. In early G1 phase, Cdc6 and cdt1 recruit Mcm2-7 onto replication origins by interacting with the origin recognition complex (ORC) to form pre-RCs. This results in origins being licensed for replication in the subsequent S phase.

HPV type 1 (HPV1) E1̂E4 inhibits initiation of DNA synthesis. Co-immunoprecipitation studies indicate that E1̂E4 may exert its inhibitory function through interaction with the replication initiation factors Cdc6 and Mcm7. Interactions between E2̂E4 and the licensing factors Cdc6 and Mcm7 may be part of a viral mechanism that results in repression of pre-RC function and thus inhibition of replication initiation. The extreme N-terminus of the E1̂E4 protein, although not required for interaction with Cdc6 and Mcm7 may have an essential function in this inhibitory mechanism. This system may be modulated in vivo by introducing entities such as HPV1 E1̂E4 into cells in a DNA synthesis phase (S-phase) assay.

In the following examples, the S-phase assay is used to demonstrate the effects of various moieties carried into the cell using SMCs according to the present invention. In this example, the S-phase assay is used to demonstrate the effects of the 125aa HPV1 E1̂E4 polypeptide carried into the cell using SMCs according to the present invention. The amino acid sequence of the HPV1 E1̂E4 peptide is shown in FIG. 4.

S-Phase Assay with Microinjection

A positive demonstration of the effect of S-phase inhibition by HPV E1̂E4 can be achieved by the microinjection of plasmid expressing HPV1 E1̂E4 (which microinjection does not form part of the present invention). Plasmids expressing E2̂E4 are microinjected into the nuclei of NIH3T3 fibroblasts at the treatment time 8 hours after release from density-dependent growth arrest in G0. The ability of cells to initiate DNA replication was assessed by pulse labelling cells 19 hours after release from G0 for 1 hour with bromodeoxyuridine (BrdU). Expression of E1̂E4 protein and incorporation of BrdU was monitored by confocal immunofluorescence microscopy. Only a small proportion of E2̂E4-expressing cells (13%), compared to non-injected cells (49%), remained competent to progress into S phase. Cells expressing E1̂E4 and synthesising DNA exhibited less intense BrdU-specific nuclear fluorescence, suggesting that the quantity of E1̂E4 in these cells may have been insufficient to completely inhibit replication. Cells microinjected with a plasmid expressing an irrelevant control protein (a fusion protein between the small GTPase ADP-ribosylation factor ARF and cyan fluorescent protein) initiated DNA replication at a frequency indistinguishable from non-injected cells. Taken together, these data demonstrate that expression of HPV1 E1̂E4 in synchronised G1 phase NIH3T3 fibroblasts inhibits entry into S phase.

S-Phase Assay with SMC-Delivered HPV E1̂E4 Peptide

A functional demonstration of the invention is provided by introduction of the HPV E1̂E4 polypeptide into the cells by conjugation to a SMC according to the present invention. In this example, the HPV E1̂E4 peptide is coupled/conjugated by reacting:
(i) a compound of formula Ic, or a pharmaceutically acceptable salt thereof, as defined hereinbefore; and
(ii) a 125 amino acid HPV1 E1̂E4 polypeptide.

The treatment takes place at 8 hours after G0 release.

A final concentration of 10 fM to 10 μM of the conjugate is contacted with the cells.

The SMC-HPV1E1̂E4 is efficiently taken into the cells and inhibits DNA synthesis as monitored by the above assay.

### Example 6

This is performed as in Example 5 except that the cargo (ii) is the pre-RC assembly repressor protein Geminin (209 aa). The amino acid sequence for Geminin is shown in FIG. 5.

The treatment takes place at 8 hours after G0 release.

A final concentration of 10 fM to 10 μM of the conjugate is contacted with the cells. The SMC-Geminin is efficiently taken into the cells and inhibits DNA synthesis as monitored by the above assay. A dramatic reduction in the number of cells entering S phase is observed.

### Example 7

Dbf4/ASK recruits Cdc7 to the pre-RC which in turn is essential for origin firing which leads to unwinding of the DNA helix. In this example, the effects of these interactions are disrupted using SMC-cargo according to the present invention.

This is performed as in Example 5 except that the cargo (ii) is a small peptide based on the Dbf4/ASK regulator for Cdc7 kinase activity. The amino acid sequence of this peptide is shown in FIG. 6. This peptide competes with endogenous Dbf4/ASK for the binding site on Cdc7 thus acting as a dominant negative.

The treatment takes place at 8 hours after G0 release.

A final concentration of 30 nM to 30 μM of the conjugate is contacted with the cells. The SMC-Dbf4 regulator is efficiently taken into the cells and inhibits DNA synthesis as monitored by the above assay.

### Example 8

This is performed as in Example 5 except that the cargo (ii) is an antibody against the pre-RC constituent Cdc6. This protein is de novo synthesised during release from G0 and is critical for pre-RC assembly and S phase entry. Treatment takes place at 8 hours after G0 release.

A final concentration of 10 mM to 10 μM of the conjugate is contacted with the cells. The SMC-anti-Cdc6 antibody is efficiently taken into the cells.

Transport of anti-Cdc6 antibody into the nucleus blocks Cdc6 function and thus entry into S phase, and inhibits DNA synthesis as monitored by the above assay.

### Example 9

This is performed as in Example 5 except that the cargo (ii) is an anti-sense oligomer targetting Cdc6 mRNA and thus blocking Cdc6 synthesis with the same effects as discussed in Example 8. The treatment takes place at 8 hours after G0 release.

A final concentration of 50 nM to 10 μM of the conjugate is contacted with the cells. The SMC-oligomer is efficiently taken into the cells and inhibits DNA synthesis as monitored by the above assay.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

1-45. (canceled)

46. A compound of formula I, or a pharmaceutically acceptable salt thereof, 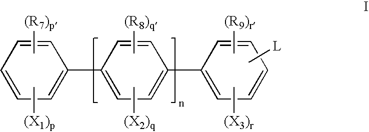
wherein
X₁, X₂ and X₃ are each independently 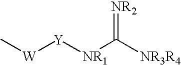
where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
R₇, R₈ and R₉ are each independently selected from H, alkyl, halo, CF₃, OH, alkoxy, NH₂, CN, NO₂ and COOH;
q and r are each independently 1, 2, 3 or 4;
q′ and r′ are each independently 0, 1, 2 or 3, where q+q′ and r+r′ each equal 4;
p is 1, 2, 3, 4 or 5, and p′ is 0, 1, 2, 3 or 4, where p+p′ is 5;
n is 0, 1, 2, 3 . . . 6;
L is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore, 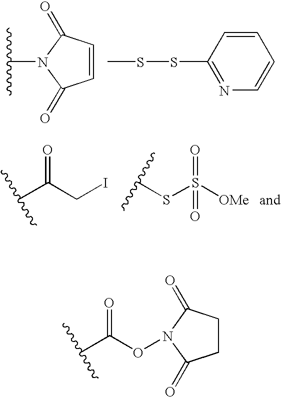
or R₅, R₆ and the nitrogen to which they are attached together form

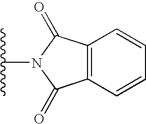

47. A compound according to claim 46 wherein Y is a C₁₋₁₀ alkylene group, a C₂₋₁₀ alkenylene group or a C₂₋₁₀ alkynylene group.

48. A compound according to claim 46 wherein W is O.

49. A compound according to claim 46 wherein Y is CH₂CH₂.

50. A compound according to claim 46 wherein Z is an alkylene group.

51. A compound according to claim 46 wherein Z is a CH₂ group.

52. A compound according to claim 46 wherein one of R₅ and R₆ is H and the other is selected from H, CO(CH₂)ⱼQ₁ or C═S(NH)(CH₂)ₖQ₂, or R₅, R₆ and the nitrogen to which they are attached together form

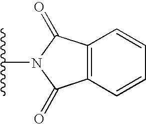

53. A compound according to claim 46 wherein L is selected from the following: CH₂NH₂, CH₂NHCOCH₂CH₂COOH

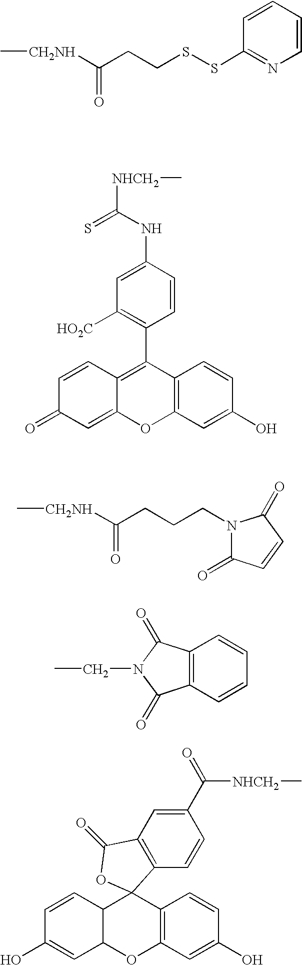

54. A compound according to claim 46 wherein R₁, R₂, R₃ and R₄ are each independently selected from H, or a butyloxycarbonyl (Boc) protecting group.

55. A compound according to claim 46 wherein p, q and r are each independently 1 or 2.

56. A compound according to claim 46 wherein X₁, X₂ and X₃ are the same and are all 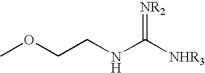
and where R₂ and R₃ are each independently H or a Boc protecting group.

57. A compound according to claim 46 wherein n is 0.

58. A compound according to claim 57 wherein said compound is of formula Ia or Ib

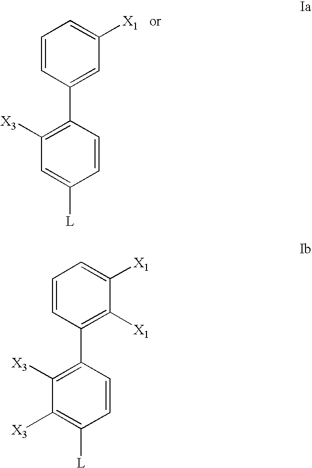

59. A compound according to claim 58 wherein X₁ and X₃ are the same and are both 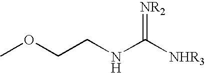
and where R₂ and R₃ are each independently H or a Boc protecting group.

60. A compound according to claim 46 which is selected from the following:

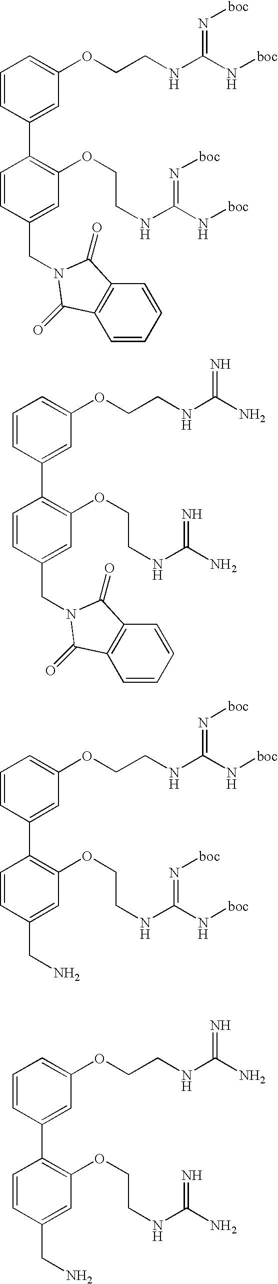

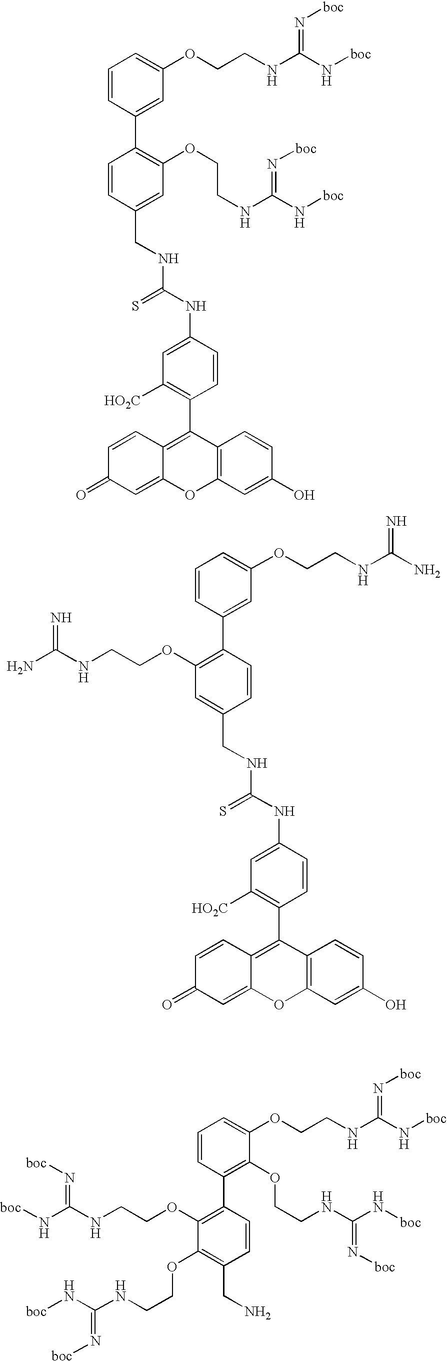

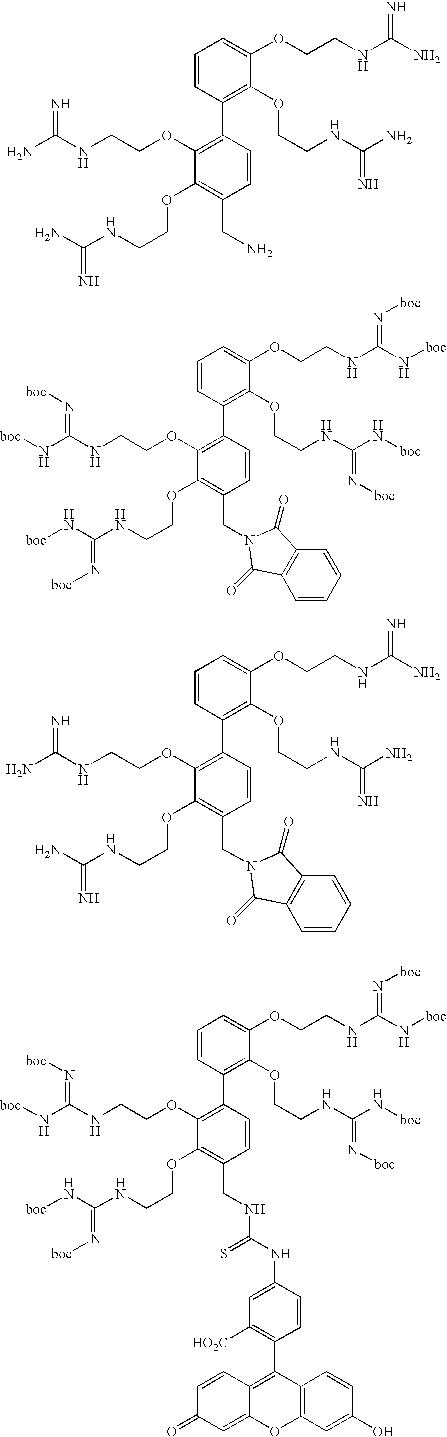

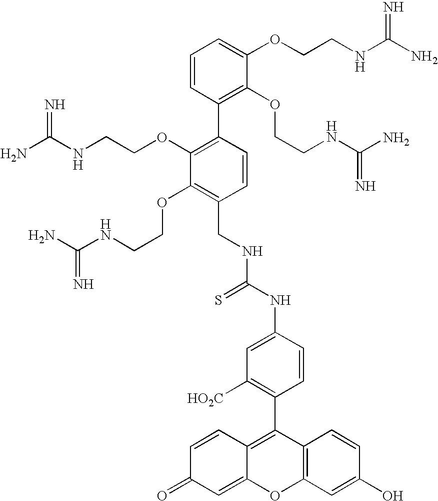

61. A conjugate comprising a compound of formula I as defined in claim 46 linked to a cargo moiety.

62. A conjugate comprising the reaction product of:
a compound of formula Ic, or a pharmaceutically acceptable salt thereof, 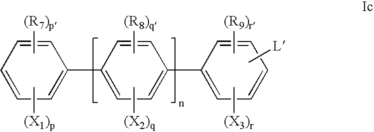
wherein
X₁, X₂ and X₃ are each independently 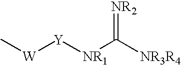
where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
R₇, R₈ and R₉ are each independently selected from H, alkyl, halo, CF₃, OH, alkoxy, NH₂, CN, NO₂ and COOH;
q and r are each independently 1, 2, 3 or 4;
q′ and r′ are each independently 0, 1, 2 or 3, where q+q′ and r+r′ each equal 4;
p is 1, 2, 3, 4 or 5, and p′ is 0, 1, 2, 3 or 4, where p+p′ is 5;
n is 0, 1, 2, 3 . . . 6;
L′ is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore 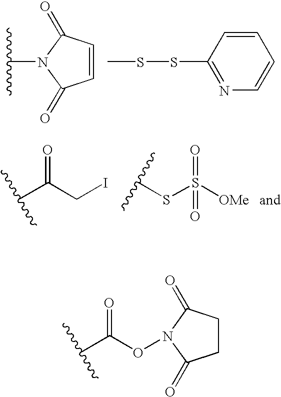
and
a cargo moiety selected from a protein, a peptide, an antibody or a drug.

63. A conjugate according to claim 61 wherein the cargo moiety is selected from a protein, a peptide, an oligonucleotide, a nucleotide, a diagnostic agent, a biologically active compound, an antibody and a drug.

64. A conjugate according to claim 61 wherein the cargo moiety is covalently attached to the L group of said compound of formula I.

65. A delivery system comprising a drug moiety linked to a carrier moiety, wherein the carrier moiety is a compound of formula I as defined in claim 46.

66. A delivery system according to claim 65, wherein the delivery system is therapeutically active in its intact state.

67. A delivery system according to claim 65, wherein the drug moiety is derived from a cytotoxic drug.

68. A delivery system according to claim 67, wherein the drug moiety is selected from DNA damaging agents, anti-metabolites, anti-tumour antibiotics, natural products and their analogues, dihydrofolate reductase inhibitors, pyrimidine analogues, purine analogues, cyclin-dependent kinase inhibitors, thymidylate synthase inhibitors, DNA intercalators, DNA cleavers, topoisomerase inhibitors, anthracyclines, vinca drugs, mitomycins, bleomycins, cytotoxic nucleosides, pteridine drugs, diynenes, podophyllotoxins, platinum containing drugs, differentiation inducers and taxanes.

69. A delivery system according to claim 68, wherein the drug moiety is selected from methotrexate, methopterin, dichloromethotrexate, 5-fluorouracil, 6-mercaptopurine, tri-substituted purines such as olomoucine, roscovitine and bohemine, flavopiridol, staurosporin, cytosine arabinoside, melphalan, leurosine, actinomycin, daunorubicin, doxorubicin, mitomycin D, mitomycin A, caminomycin, aminopterin, tallysomycin, podophyllotoxin (and derivatives thereof), etoposide, cisplatinum, carboplatinum, vinblastine, vincristine, vindesin, paclitaxel, docetaxel, taxotere retinoic acid, butyric acid, acetyl spermidine, tamoxifen, irinotecan and camptothecin.

70. A delivery system according to claim 65, wherein the drug moiety is directly linked to the carrier moiety.

71. A delivery system according to claim 65, wherein the drug moiety is indirectly linked to the carrier moiety by means of a linker moiety.

72. A delivery system according to claim 65, wherein each carrier moiety bears more than one drug moiety.

73. A delivery system according to claim 72, wherein the drug moieties are different.

74. A delivery system according claim 72, wherein each drug moiety is linked to the carrier moiety by way of a linker moiety.

75. A delivery system according to claim 74, wherein each drug moiety is linked to the carrier moiety by an identical linker moiety.

76. A delivery system according to claim 74, wherein each drug moiety is linked to the carrier moiety by a different linker moiety.

77. A delivery system according to claim 65, further comprising a targetting moiety.

78. A delivery system according to claim 77, wherein the targetting moiety is attached to the carrier moiety.

79. A delivery system according to claim 77, wherein the targetting moiety is attached to the drug moiety.

80. A compound of formula Id, or a pharmaceutically acceptable salt thereof, 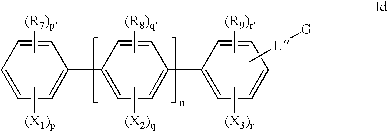
wherein
X₁, X₂ and X₃ are each independently 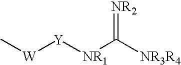
where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
R₇, R₈ and R₉ are each independently selected from H, alkyl, halo, CF₃, OH, alkoxy, NH₂, CN, NO₂ and COOH;
q and r are each independently 1, 2, 3 or 4;
q′ and r′ are each independently 0, 1, 2 or 3, where q+q′ and r+r′ each equal 4;
p is 1, 2, 3, 4 or 5, and p′ is 0, 1, 2, 3 or 4, where p+p′ is 5;
n is 0, 1, 2, 3 . . . 6;
L″ is -(Z)ₘNR₅— where Z is a hydrocarbyl group and m is 0 or 1;
where R₅ is H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore, 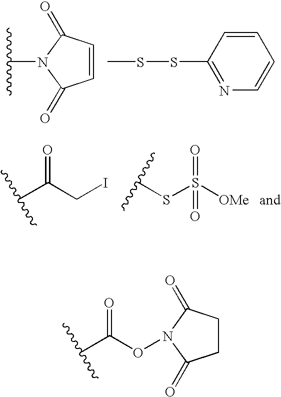
G is a cargo moiety.

81. A compound according to claim 80 wherein L″ is -(Z)ₘNH.

82. A pharmaceutical composition comprising a compound according to claim 46,
a conjugate comprising the reaction product of:
a compound of formula Ic, or a pharmaceutically acceptable salt thereof, 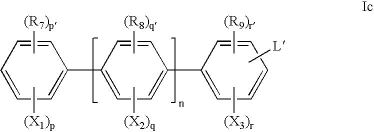
wherein
X₁, X₂ and X₃ are each independently 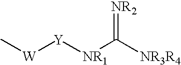
where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
R₇, R₈ and R₉ are each independently selected from H, alkyl, halo, CF₃, OH, alkoxy, NH₂, CN, NO₂ and COOH;
q and r are each independently 1, 2, 3 or 4;
q′ and r′ are each independently 0, 1, 2 or 3, where q+q′ and r+r′ each equal 4;
p is 1, 2, 3, 4 or 5, and p′ is 0, 1, 2, 3 or 4, where p+p′ is 5;
n is 0, 1, 2, 3 . . . 6;
L′ is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore 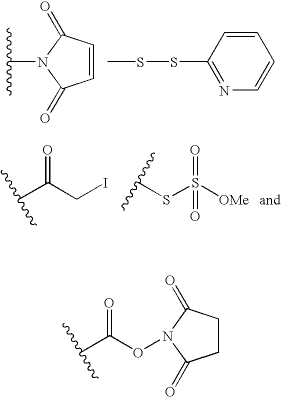
and
a cargo moiety selected from a protein, a peptide, an antibody or a drug,
or a delivery system comprising a drug moiety linked to a carrier moiety, wherein the carrier moiety is a compound of formula I,
and a pharmaceutically acceptable excipient, diluent or carrier.

83. A compound according to claim 46, a conjugate comprising the reaction product of:
a compound of formula Ic, or a pharmaceutically acceptable salt thereof, 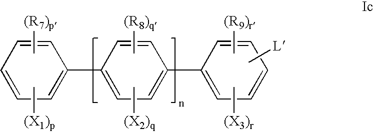
wherein
X₁, X₂ and X₃ are each independently 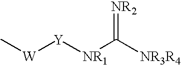
where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
R₇, R₈ and R₉ are each independently selected from H, alkyl, halo, CF₃, OH, alkoxy, NH₂, CN, NO₂ and COOH;
q and r are each independently 1, 2, 3 or 4;
q′ and r′ are each independently 0, 1, 2 or 3, where q+q′ and r+r′ each equal 4;
p is 1, 2, 3, 4 or 5, and p′ is 0, 1, 2, 3 or 4, where p+p′ is 5;
n is 0, 1, 2, 3 . . . 6;
L′ is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, a chromophore 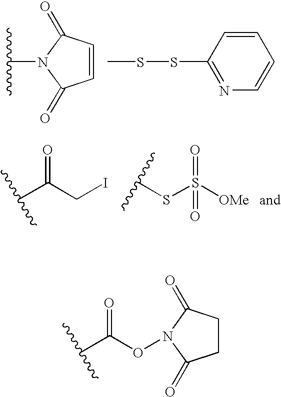
and
a cargo moiety selected from a protein, a peptide, an antibody or a drug,
or a delivery system comprising a drug moiety linked to a carrier moiety, wherein the carrier moiety is a compound of formula I,
for use in medicine.

84. A process for preparing a conjugate, said process comprising reacting a compound of formula Ic, or a pharmaceutically acceptable salt thereof, 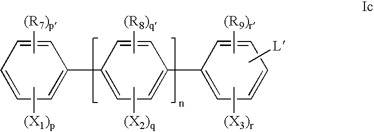
wherein
X₁, X₂ and X₃ are each independently 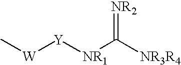
where Y is an alkylene, alkenylene or alkynylene group, each of which may be optionally substituted with one or more substituents selected from alkyl, halo, CF₃, OH, alkoxy, NH₂, CN, NO₂ and COOH;
W is absent or is O, S or NH;
R₁, R₂, R₃ and R₄ are each independently selected from H, alkyl, aryl and a protecting group P₁;
R₇, R₈ and R₉ are each independently selected from H, alkyl, halo, CF₃, OH, alkoxy, NH₂, CN, NO₂ and COOH;
q and r are each independently 1, 2, 3 or 4;
q′ and r′ are each independently 0, 1, 2 or 3, where q+q′ and r+r′ each equal 4;
p is 1, 2, 3, 4 or 5, and p′ is 0, 1, 2, 3 or 4, where p+p′ is 5;
n is 0, 1, 2, 3 . . . 6;
L′ is (Z)ₘNR₅R₆ where Z is a hydrocarbyl group and m is 0 or 1;
where R₅ and R₆ are each independently H, CO(CH₂)ⱼQ₁ or C═S(NH)(CH₂)ₖQ₂ where j and k are each independently 0, 1, 2, 3, 4 or 5, and Q₁ and Q₂ are each independently selected from COOH, 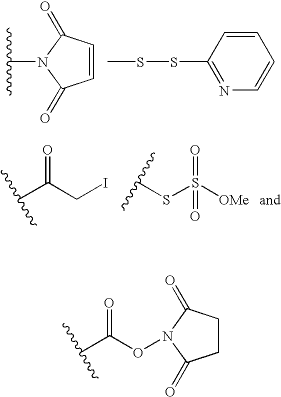
with a cargo moiety selected from a protein, a peptide, an antibody and a drug.

85. A method for introducing a cargo moiety into a cell, said method comprising contacting said cell with a conjugate according to claim 61.

86. A process for preparing a compound of formula I as defined in claim 46, said process comprising the steps of:
reacting a compound of formula II with TsOCH₂CH₂NHBoc to form a compound of formula III, where L′″ is (Z)ₘNR₅R₆ and R₅ and R₆ are an NH₂ protecting group; 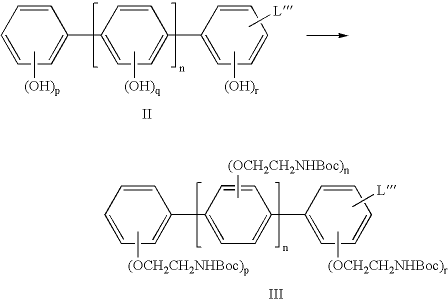
removing the Boc groups from said compound of formula III to form a compound of formula IV; 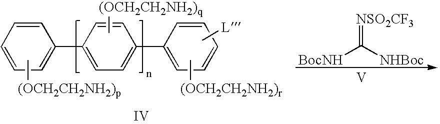
and
reacting said compound of formula IV with a compound of formula V to form a compound of formula I.

87. A process for preparing a compound of formula I as defined in claim 46, said process comprising the steps of:
reacting a compound of formula II with ClCH₂CN to form a compound of formula VI; 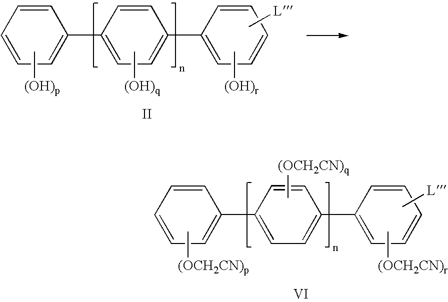
reacting said compound of formula VI with Pd/H₂/carbon to form a compound of formula IV; and 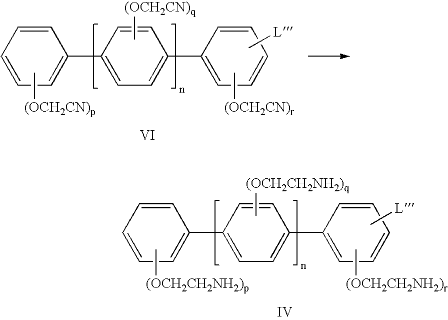
reacting said compound of formula IV with a compound of formula V to form a compound of formula I.

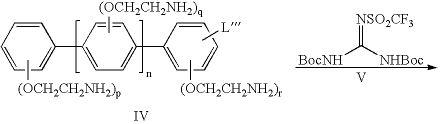

88. A process according to claim 86 wherein said compound of formula II is prepared by the steps of:
reacting a compound of formula VI with a compound of formula VII, 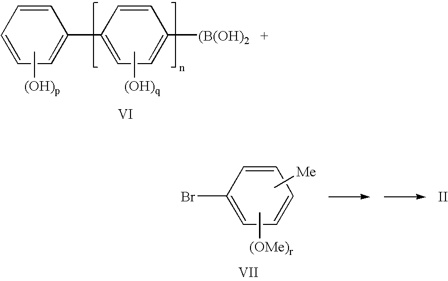
converting the product obtained from step (i) to a compound of formula II.

89. A compound of formula If, or a pharmaceutically acceptable salt thereof, 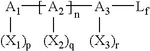
If
herein X₁, X₂, X₃, p, q, r and n are as defined in claim 46;
each of A₁, A₂ and A₃ is independently a phenyl group optionally substituted by one or more additional substituents selected from alkyl, halo, CF₃, OH, alkoxy, NH₂, CN, NO₂ and COOH; and
L_{f} is a linker group.
